(19) United States
(12) Patent Application Publication
(10) Pub. No.: US 2026/0301928 A1
(43) Pub. Date: Oct. 1, 2026

(54) SYSTEM AND METHOD FOR OPTIMIZING HEALTHCARE LOGISTICS

(71) Applicant: Pulse Charter Connect Inc., Chicago, IL (US)

(72) Inventors: Laura Epstein, Chicago, IL (US); Mark Engelen, St Petersburg, FL (US); Chris Tyler, Grapevine, TX (US)

(73) Assignee: Pulse Charter Connect Inc., Chicago, IL (US)

(21) Appl. No.: 19/629,544

(22) Filed: Mar. 26, 2026

Related U.S. Application Data
(60) Provisional application No. 63/780,134, filed on Mar. 28, 2025.

(51) Int. Cl. G16H 40/20; G01C 21/34; G06Q 10/0833

(52) U.S. Cl. CPC G16H 40/20; G01C 21/3407; G06Q 10/0833

(57) ABSTRACT

A system for organ transport logistics comprises a central server configured to coordinate organ transport operations between transplant centers and transportation providers, a storage machine coupled to the central server and configured to store transport request data, proposal data, and historical transport data in a relational database, and a frontend user interface retrievable by users. The frontend user interface is configured to receive transport request inputs from transplant coordinators and display proposal information from transportation providers. The central server is configured to receive transport request data comprising organ type, recovery location, recipient facility, and timing requirements, transmit the transport request data to transportation providers based on matching criteria, receive and aggregate proposal data, receive a selection input indicating acceptance of a selected proposal, retrieve real-time location data from nodes along an organ transport chain, and generate notifications to members of the organ transport chain.

### CROSS-REFERENCE TO RELATED APPLICATIONS

This application claims priority to U.S. Application No. 63/780,134, titled SYSTEM AND METHOD FOR OPTIMIZING HEALTHCARE LOGISTICS, filed Mar. 28, 2025, which is hereby incorporated by reference in its entirety.

### FIELD OF INVENTION

The disclosure relates to the field of healthcare logistics, and more specifically to systems, methods, and devices for optimizing and automating the process of organ transport logistics. In particular, the present disclosure pertains to digital Software as a Service (Saas) solutions that facilitate the coordination of transportation resources, enable real-time communication between transplant centers and charter operators, and provide comprehensive tracking and analytics capabilities for time-sensitive medical shipments.

### BACKGROUND

In the field of healthcare logistics, particularly in the context of organ transplantation, the process of transporting organs from donors to recipients is of paramount concern. The logistics involved in this process are complex and multifaceted, involving numerous stakeholders, including transplant centers, charter operators, and healthcare professionals. The process typically involves the coordination of transportation resources, including planes and pilots, to ensure that organs are delivered in a timely and efficient manner.

Transplant coordinators play a coordinating role in this process, as they are responsible for communicating with the relevant parties, managing invoicing, and aggregating performance metrics. They are often tasked with making decisions on a case-by-case basis, considering various factors such as the availability of transportation resources, the urgency of the transplant, and the viability of the organ.

Given the time-sensitive nature of organ transplantation, the ability to monitor shipments in real-time is a useful asset. This involves tracking the location of the organ during transit and ensuring its safety throughout the journey. Additionally, the ability to analyze historical transport data can provide useful insights for future transportation planning and decision-making.

Furthermore, the use of digital platforms and software as a service (SaaS) solutions is becoming increasingly prevalent in the healthcare sector. These technologies offer the potential to automate and streamline various aspects of healthcare logistics, thereby enhancing efficiency and reducing costs.

However, existing approaches to organ transport logistics present substantial challenges that can impact patient outcomes. The coordination of multiple transportation providers, including air charter operators and ground transportation services, often relies on fragmented communication channels that can lead to delays and miscommunication during time-sensitive operations. Transplant coordinators frequently may need to contact multiple charter companies individually to obtain quotes and availability information, a process that consumes limited time when organs have limited viability windows. The lack of centralized visibility into the transportation process means that transplant centers may have difficulty tracking the precise location and status of organs in transit, creating uncertainty during surgical preparation. Additionally, the absence of standardized mechanisms for aggregating and analyzing historical transport data limits the ability of healthcare organizations to identify inefficiencies, benchmark performance, and make data-driven decisions regarding transportation preferences. The manual nature of invoicing and performance metric collection further burdens transplant coordinators, diverting their attention from patient care responsibilities.

Despite some advancements, the field of healthcare logistics, particularly in the context of organ transplantation, continues to evolve, with ongoing efforts to improve the efficiency, safety, and success rates of organ transport.

Existing systems for coordinating organ transport rely heavily on manual processes that introduce friction and delay into time-sensitive operations. Under the current model, when a transplant center requires air transportation for an organ recovery, coordinators may need to individually contact multiple charter operators by telephone or email, often without any guarantee of receiving a timely response. This manual outreach process is inherently inefficient, as coordinators may spend limited time waiting for callbacks or chasing down availability information from operators who may ultimately be unable to fulfill the request. The lack of automation means that coordinators may need to personally manage the filtering and comparison of options, a cognitive burden that diverts attention from clinical responsibilities. An improved system that automates the request for proposal process and implements a filter and prioritization matrix could eliminate much of this manual work, enabling transport requests to be distributed to qualified operators simultaneously and proposals to be aggregated and ranked without requiring individual follow-up by the coordinator. Such automation could return results with high velocity, compressing the time between transport request submission and proposal selection, thereby preserving precious minutes during the organ viability window and potentially improving patient outcomes.

### SUMMARY

This summary is provided to introduce a selection of concepts in a simplified form that are further described below in the detailed description. This summary is not intended to identify particular features or core features of the claimed subject matter, nor is it intended to be used as an aid in determining the scope of the claimed subject matter.

The instant disclosure in one form is directed to systems, methods, and devices for optimizing and automating the process of organ transport logistics.

This summary is provided to introduce a selection of concepts in a simplified form that are further described below in the detailed description. This summary is not intended to identify particular features or core features of the claimed subject matter, nor is it intended to be used as an aid in determining the scope of the claimed subject matter.

According to an aspect of the present disclosure, a digital SaaS solution for Critical Healthcare Logistics includes a platform for automating the process of organ transport logistics, a system for providing transplant centers with dedicated access to planes and pilots, a feature for enabling transplant coordinators to communicate directly with the relevant parties, a mechanism for streamlining invoicing and aggregating performance metrics, and a function for increasing transparency throughout the supply chain.

According to other aspects of the present disclosure, the digital SaaS solution may include an integrated chat functionality enabling secure direct messaging between all users in real-time, which may be used to discuss time-sensitive organ assignments, resolve transport issues, and get assistance. The solution may also include a dashboard providing users with visual analytics such as maps and insights on completed organ transport trips, which transforms historical transport data into interactive graphs and metrics, like cost percentages and time estimates. Additionally, the solution may include a flight tracking feature allowing transplant centers to monitor shipments in real-time, which compiles shipment routes over time, building historical tracking data that Transplant Centers can analyze to inform future transportation preferences and decision making.

According to another aspect of the present disclosure, a method for optimizing healthcare logistics includes creating and submitting transport requests detailing transportation requirements through a frontend user interface, receiving and reviewing proposals from Charter Operators, selecting a preferred proposal and confirming the same, consolidating charter details in a centralized hub, and providing access to historical transport data and invoicing.

According to other aspects of the present disclosure, the method may include an integrated chat functionality enabling secure direct messaging between all users in real-time, which is used to discuss time-sensitive organ assignments, resolve transport issues, and get assistance. The method may also include a dashboard providing users with visual analytics and insights on completed organ transport trips, which transforms historical transport data into interactive graphs and metrics, like cost percentages and time estimates. Additionally, the method may include a flight and/or ground tracking feature allowing Transplant Centers to monitor shipments in real-time, which compiles shipment routes over time, building historical tracking data that Transplant Centers can analyze to inform future transportation preferences and decision making. The dashboard may further include a map detailing the transport route. In other embodiments, the map may show alternative routes.

According to yet another aspect of the present disclosure, a system for streamlining organ transport logistics includes an integrated chat functionality for real-time communication, a dashboard providing visual analytics and insights on completed organ transport trips, a flight tracking feature for real-time monitoring of shipments, a shipment reminder system for sending automated notifications, and a backend hosted on AWS using a relational database (e.g., a Postgres database).

According to other aspects of the present disclosure, the system may include an integrated chat functionality that includes a feature for secure direct messaging between transplant coordinators, charter operators, and help desk staff, which is used to discuss time-sensitive organ assignments, resolve transport issues, and get assistance. The system may also include a dashboard that transforms historical transport data into interactive graphs and metrics, like cost percentages and time estimates.

Additionally, the system may include a flight tracking feature that may compile shipment routes over time, building historical tracking data that Transplant Centers may analyze to inform future transportation preferences and decision making. Various ground and air transportation methods such as airplane, helicopter, automobile, truck, bus or rail may also be integrated into the system of the instant disclosure. Furthermore, the system may include a shipment reminder system that may automatically send notifications to relevant users at specified intervals throughout the organ transportation process.

According to an aspect of the present disclosure, a system for organ transport logistics may be provided. The system may include a central server configured to coordinate organ transport operations between transplant centers and transportation providers. The system may include a storage machine coupled to the central server and configured to store transport request data, proposal data, and historical transport data in a relational database. The system may include a frontend user interface retrievable by users of the system, the frontend user interface configured to receive transport request inputs from transplant coordinators and display proposal information from transportation providers. The central server may be configured to receive transport request data from the frontend user interface, the transport request data comprising organ type, recovery location, recipient facility, and timing parameters. The central server may be configured to transmit the transport request data to a plurality of transportation providers based on matching criteria. The central server may be configured to receive proposal data from the plurality of transportation providers, the proposal data comprising pricing information, vehicle specifications, and estimated travel times. The central server may be configured to aggregate the proposal data and transmit the aggregated proposal data to the frontend user interface for display to the transplant coordinators. The central server may be configured to receive a selection input indicating acceptance of a selected proposal. The central server may be configured to retrieve real-time location data from nodes along an organ transport chain during transport operations. The central server may be configured to generate notifications to members of the organ transport chain based on the real-time location data.

According to other aspects of the present disclosure, the system may include one or more of the following features. The central server may further comprise a scheduling module configured to match transport parameters with available transportation resources based on factors comprising availability of aircraft, urgency of the organ transport, distance between the recovery location and the recipient facility, and weather conditions. The scheduling module may be further configured to generate route recommendations based on routing optimization algorithms that may analyze transplant center preferences, origin and destination locations, distances, weather data, traffic conditions, and transportation operator availability. The central server may further comprise a tracking module configured to compile shipment routes over time and store the compiled routes as historical tracking data in the relational database for analysis by transplant centers to inform future transportation preferences. The tracking module may be configured to retrieve real-time location data from nodes along the organ transport chain comprising ground transportation vehicles, aircraft, and donor facilities. The central server may further comprise a chat module configured to enable secure direct messaging between transplant coordinators, charter operators, drivers, pilots, and help desk staff in real-time during organ transport operations. The central server may further comprise an insights module configured to transform the historical transport data into interactive graphs and metrics comprising cost percentages, time estimates, and performance benchmarks for display through the frontend user interface. The central server may further comprise an invoicing module configured to automate generation of invoices based on completed transport operations and aggregate financial data comprising billing information and payment records in the relational database.

According to another aspect of the present disclosure, a method for coordinating organ transport logistics may be provided. The method may include receiving, by a central server, transport request data from a user interface, the transport request data comprising organ type, recovery location, recipient facility, and timing parameters. The method may include matching, by the central server, the transport request data to a plurality of transportation providers based on matching criteria stored in a relational database, wherein the matching criteria are applied through matching algorithms that evaluate transplant center preferences, safety parameters, location proximity, and transportation operator availability. The method may include transmitting, by the central server, the transport request data to the matched plurality of transportation providers. The method may include receiving, by the central server, proposal data from the plurality of transportation providers, the proposal data comprising pricing information and estimated travel times. The method may include aggregating, by the central server, the proposal data and transmitting the aggregated proposal data to the user interface. The method may include receiving, by the central server, a selection input indicating acceptance of a selected proposal from the aggregated proposal data. The method may include retrieving, by the central server, real-time location data from nodes along an organ transport chain during transport operations. The method may include generating, by the central server, notifications to members of the organ transport chain based on the retrieved real-time location data.

According to yet another aspect of the present disclosure, a system for dynamically matching and optimizing transport resources for time-sensitive logistics missions may be provided. The system may include a central server comprising a request intake module configured to receive mission-specific inputs through a frontend user interface, the mission-specific inputs comprising at least origin location, destination location, timing constraints, and cargo specifications. The central server may comprise a supply availability module configured to maintain a registry of available transportation resources and operators within a relational database, the registry comprising resource specifications, location data, and availability status. The central server may comprise a telemetry ingestion module configured to ingest real-time operational data from external sources, the real-time operational data comprising location data and status indicators. The central server may comprise a pricing engine configured to generate dynamic price estimates based on a pricing model. The central server may comprise a feasibility engine configured to evaluate whether a mission can be executed based on constraint categories (comprising aircraft compatibility constraints, operational constraints, and clinical timing constraints). The central server may comprise an optimization and ranking engine configured to generate ranked transport recommendations based on configurable weighting factors. The system may include a storage machine coupled to the central server and configured to store transport data in the relational database. The system may include the frontend user interface retrievable by users of the system, the frontend user interface configured to receive the mission-specific inputs and display the ranked transport recommendations.

According to other aspects of the present disclosure, the system may include one or more of the following features. The central server may further comprise a mobile device integration module configured to ingest mobile device data from electronic devices associated with participants in a transport chain, the mobile device data comprising location data and timing information. The pricing engine may be configured to apply a baseline activation model to determine a minimum price specified to activate a transportation resource, apply an incremental cost model based on mission duration parameters, and apply a dynamic adjustment model based on operational conditions. The feasibility engine may be configured to perform compatibility analysis comprising capacity assessments and operational constraint evaluations based on transportation resource capability data stored in the relational database. The central server may further comprise a real-time re-optimization module configured to monitor event streams and, upon detection of a triggering event, invoke the pricing engine, feasibility engine, and optimization and ranking engine to generate updated recommendations. The central server may further comprise a learning and feedback module configured to compare predicted outcomes against actual outcomes recorded upon completion of transport operations, and update system parameters based on historical performance data stored in the relational database.

According to other aspects of the present disclosure, the method may include one or more of the following features. Matching the transport request data to the plurality of transportation providers may comprise applying routing optimization algorithms that may analyze transplant center preferences, origin and destination locations, distances, weather data, traffic conditions, and transportation operator availability to generate route recommendations. The method may further comprise compiling shipment routes over time and storing the compiled routes as historical tracking data in the relational database for analysis by transplant centers to inform future transportation preferences. The method may further comprise enabling secure direct messaging between transplant coordinators, charter operators, drivers, pilots, and help desk staff in real-time during the transport operations through a chat module of the central server. The method may further comprise transforming the historical transport data into interactive graphs and metrics comprising cost percentages, time estimates, and performance benchmarks, and transmitting the interactive graphs and metrics to the user interface for display. The method may further comprise automating generation of invoices based on completed transport operations and aggregating financial data comprising billing information and payment records in the relational database.

These and other objects, features, and advantages of the present disclosure will become more readily apparent from the attached drawings and the detailed description of the preferred embodiments, which follow.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 is a database diagram of the system in accordance with one or more embodiments of the present disclosure;

FIG. 2 is an illustration of a user interface screen for a transportation request in accordance with one or more embodiments of the present disclosure;

FIG. 3 is an illustration of a UI screen for a messaging feature of the system in accordance with one or more embodiments of the present disclosure;

FIG. 4 is an illustration of a UI screen for a pending transportation request in accordance with one or more embodiments of the present disclosure;

FIG. 5 is an illustration of an example use-case scenario in accordance with one or more embodiments of the present disclosure;

FIG. 6 is an illustration of a communication structure in accordance with one or more embodiments of the present disclosure;

FIG. 7 is a flowchart for a method for receiving and managing transportation requests in accordance with one or more embodiments of the present disclosure;

FIG. 8 is a flowchart for a method depicting ground transportation coordination steps for organ transport logistics in accordance with one or more embodiments of the present disclosure;

FIG. 9 is a flowchart for a method for coordinating organ transport logistics in accordance with one or more embodiments of the present disclosure; and

FIG. 10 is a flowchart for a method for communication between a transplant center team and an internal team for the transportation of materials in accordance with one or more embodiments of the present disclosure.

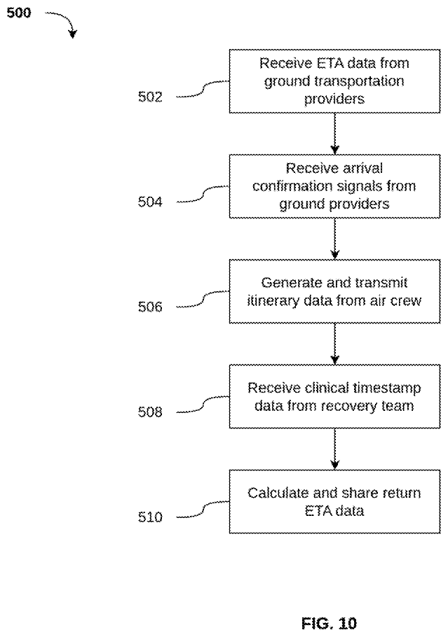

Corresponding reference characters indicate corresponding parts throughout the several views. The exemplifications set out herein illustrate embodiments of the disclosure and such exemplifications are not to be construed as limiting the scope of the disclosure in any manner.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the disclosure.

In the following description, for the purposes of explanation, numerous specific details are set forth in order to provide a thorough understanding of the described embodiments. It will be apparent to one skilled in the art however that other embodiments of the present disclosure may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the disclosure, as other embodiments of the disclosure may omit such features.

In this application the use of the singular includes the plural unless specifically stated otherwise and use of the terms “and” and “or” is equivalent to “and/or,” also referred to as “non exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, Band/or C” mean “any of the following: A; B; C; A and B; A and C; B and C; A, B and C.” An exception to this definition will occur only when a combination of elements, functions, steps or acts are in some way inherently mutually exclusive.

As this disclosure is susceptible to embodiments of many different forms, it is intended that the present disclosure be considered as an example of the principles of the disclosure and not intended to limit the disclosure to the specific embodiments shown and described.

The following description sets forth exemplary aspects of the present disclosure. It should be recognized, however, that such a description is not intended as a limitation on the scope of the present disclosure. Rather, the description also encompasses combinations and modifications to those exemplary aspects described herein.

The present disclosure relates to a digital Software as a Service (SaaS) solution for optimizing healthcare logistics, particularly in the field of organ transport. In some aspects, the solution may provide a platform for automating the process of organ transport logistics, a system for providing transplant centers with dedicated access to planes and pilots, and a feature for enabling transplant coordinators to communicate directly with the relevant parties. Various ground transportation methods and air transportation methods such as airplane, helicopter, automobile, truck, bus or rail can also be integrated into the system of the instant disclosure and the system would incorporate the associated personnel for operating the various methods of transportation. Further, the solution may include a mechanism for streamlining invoicing and aggregating performance metrics, and a function for increasing transparency throughout the supply chain.

In some aspects, the system and methods herein may democratize access to organ transportation services by expanding the network of qualified transportation providers beyond traditional charter operators. The system may enable a broader range of transportation companies and individual operators to participate in organ transport logistics by maintaining comprehensive records of their capabilities, certifications, locations, and availability within the relational database. Transportation providers that previously could not participate in organ transport operations due to lack of visibility or connectivity with transplant centers may now register with the platform and receive transport requests matched to their specific qualifications and geographic coverage. The central server may store and continuously update transportation provider profiles comprising vehicle specifications, crew certifications, operational history, safety records, and real-time availability status, thereby enabling the matching algorithms to identify suitable providers from an expanded pool of qualified operators. This democratization of access may potentially increase the availability of transportation resources for time-critical organ transport operations, reduce costs through increased competition, and improve geographic coverage in regions previously underserved by traditional charter networks.

In other aspects, the expansion of the transportation provider network may be facilitated by the tracking module and communication subsystem of the central server, which may maintain real-time visibility into the location, status, and capacity of all registered transportation providers. The system may receive GPS data and availability signals from electronic devices associated with pilots, drivers, and fleet operators, enabling the platform to identify transportation resources that are positioned to respond quickly to urgent organ transport requests. By aggregating this information in the relational database and making it accessible through the frontend user interface, the system and methods herein may enable transplant coordinators to access a comprehensive marketplace of transportation options that was previously fragmented and difficult to navigate. The proprietary matching algorithms may evaluate transportation provider profiles against transport request parameters to generate ranked recommendations, potentially connecting transplant centers with qualified operators that would not have been discoverable through traditional communication channels.

In some aspects, the central server may integrate multiple data sources and evaluation criteria into a prioritization matrix for ranking transportation provider proposals and generating route recommendations. The scheduling module may retrieve and analyze weather data from external meteorological services, safety notification feeds from aviation and transportation authorities, cargo capacity specifications from transportation provider profiles, crew certification records, and real-time crew availability indicators to generate comprehensive assessments of each transportation option. The prioritization matrix may apply configurable weighting factors and threshold parameters that reflect transplant center preferences and regulatory specifications, ensuring that factors such as deliverability, safety, and reliability may be appropriately balanced against pricing considerations. The routing optimization algorithms may evaluate multiple potential routes and transportation mode combinations, filtering options that fail to meet minimum safety thresholds or timing parameters before presenting ranked recommendations to transplant coordinators through the frontend user interface.

In other aspects, the prioritization matrix implemented by the central server may ensure that price does not automatically determine the selection of transportation providers, recognizing that organ viability and patient outcomes may depend substantially on reliable and safe delivery. The insights module may transform historical performance data into metrics that inform the weighting factors applied within the prioritization matrix, enabling the system to learn from past transport operations and refine its recommendations over time. The scheduling module may apply optimization algorithms that analyze operating room schedules, organ viability windows, and estimated travel times to identify transportation options that may maximize the probability of successful organ delivery within specified timeframes. By integrating crew availability data, weather forecasts, traffic conditions, and equipment specifications into a unified decision framework, the central server may generate recommendations that balance multiple competing objectives and present transplant coordinators with transportation options ranked according to their overall suitability for the specific organ transport operation.

More specifically, the digital SaaS solution of the present disclosure may include an integrated chat functionality enabling secure direct messaging between all users in real-time.

This feature may be used to discuss time-sensitive organ assignments, resolve transport issues, and get assistance. The solution may also comprise a dashboard providing users with visual analytics and insights on completed organ transport trips. The dashboard may transform historical transport data into interactive graphs and metrics, like cost percentages and time estimates, thereby providing useful insights for decision making. The dashboard may further include a map detailing the transport route. In other embodiments, the map may show alternative routes.

In addition, the digital SaaS solution may include a flight tracking feature allowing Transplant Centers to monitor shipments in real-time. This feature may compile shipment routes over time, building historical tracking data that Transplant Centers can analyze to inform future transportation preferences and decision making. Furthermore, the solution may include a shipment reminder system that automatically sends notifications to relevant users at specified intervals throughout the organ transportation process. This feature may help ensure timely and efficient organ transport, thereby potentially improving patient outcomes.

The present disclosure also relates to a method for optimizing healthcare logistics. The method may involve creating and submitting Transport Requests detailing transportation requirements through an application, receiving and reviewing proposals from Charter Operators, selecting a preferred proposal and confirming the same, consolidating charter details in a centralized hub, and providing access to historical transport data and invoicing. This method may streamline the process of organ transport logistics, thereby potentially reducing costs and improving efficiency.

Furthermore, the present disclosure relates to a system for streamlining organ transport logistics. The system may include an integrated chat functionality for real-time communication, a dashboard providing visual analytics and insights on completed organ transport trips, a flight tracking feature for real-time monitoring of shipments, a shipment reminder system for sending automated notifications, and a central server hosted on AWS using a relational database (e.g., a Postgres database). This system may provide a comprehensive solution for managing organ transport logistics, thereby potentially improving the efficiency and effectiveness of organ transport. The dashboard may further include a map detailing the transport route. In other embodiments, the map may show alternative routes.

In some aspects, the present disclosure relates to a digital SaaS solution for Critical Healthcare Logistics. This solution may include a platform for automating the process of organ transport logistics. The platform may facilitate the coordination of various aspects of organ transport, such as scheduling, tracking, and communication. This automation may potentially reduce manual errors, increase efficiency, and streamline the overall process of organ transport logistics.

In some cases, the digital SaaS solution may include an integrated chat functionality. This functionality may enable secure direct messaging between all users in real-time. For instance, transplant coordinators, charter operators, and help desk staff may use this functionality to discuss time-sensitive organ assignments, resolve transport issues, and get assistance. This real-time communication may potentially improve the coordination of organ transport and reduce delays.

In other aspects, the system may include an integrated chat functionality for real-time communication. This functionality may facilitate direct and secure messaging between all users. The real-time communication may allow for immediate resolution of issues, timely updates, and efficient coordination of organ transport logistics. This integrated chat functionality may potentially enhance the overall efficiency and effectiveness of the system.

In some aspects, the digital SaaS solution may provide transplant centers with dedicated access to planes and pilots or other types of transportation and their respective operators. This system may include a dedicated platform or interface that allows transplant centers to directly access and coordinate with a network of planes and pilots. The network may include various types of aircraft and pilots with different qualifications and availability. The dedicated access may allow transplant centers to quickly and efficiently arrange for the transportation of organs, potentially reducing the time between organ retrieval and transplantation.

In some cases, the system may include a scheduling feature that allows transplant centers to schedule flights with the available planes/helicopters and pilots. Various ground transportation methods such as automobile, truck, bus or rail can also be integrated into the system of the instant disclosure and the system would incorporate the associated personnel for operating the various methods of transportation. The scheduling feature may consider various factors, such as the availability of planes and pilots, the urgency of the organ transport, the distance between the organ retrieval and transplantation locations, and the weather conditions. This scheduling feature may potentially improve the efficiency and reliability of organ transport logistics.

In other aspects, the system may include a matching algorithm that matches the transport requirements of the transplant centers with the available planes and pilots. The matching algorithm may consider various factors, such as the size and weight of the organ, the distance and duration of the flight, and the availability and capabilities of the planes and pilots. This matching algorithm may potentially optimize the allocation of resources and improve the efficiency of organ transport logistics.

In some instances, the system may provide transplant centers with real-time updates on the status of the planes and pilots. These updates may include information such as the location of the plane, the estimated time of arrival, and any changes in the flight plan. These real-time updates may potentially increase the transparency of the organ transport process and allow transplant centers to make informed decisions.

In some aspects, the digital SaaS solution may include a feature for enabling transplant coordinators to communicate directly with the relevant parties. This feature may include an integrated chat functionality that allows secure direct messaging between all users in real-time. The integrated chat functionality may be used to discuss time-sensitive organ assignments, resolve transport issues, and get assistance. This direct communication may potentially improve the coordination of organ transport logistics and reduce delays.

In some cases, the method for optimizing healthcare logistics may also include an integrated chat functionality. This functionality may enable secure direct messaging between all users in real-time. The integrated chat functionality may be used to discuss time-sensitive organ assignments, resolve transport issues, and get assistance. This real-time communication may potentially improve the efficiency and effectiveness of the method.

In other aspects, the integrated chat functionality may include a feature for secure direct messaging between transplant coordinators, charter operators, and help desk staff. This feature may facilitate direct and secure communication between these parties, potentially improving the coordination and efficiency of organ transport logistics.

In some instances, the integrated chat functionality may be used to discuss time-sensitive organ assignments, resolve transport issues, and get assistance. This functionality may provide a platform for real-time communication and collaboration, potentially improving the efficiency and effectiveness of organ transport logistics.

In some aspects, the digital SaaS solution may include a mechanism for streamlining invoicing and aggregating performance metrics. This mechanism may automate the process of generating and sending invoices to the relevant parties, such as the transplant centers and charter operators. The automation of invoicing may potentially reduce manual errors, save time, and improve the efficiency of the billing process.

In some cases, the mechanism may include a feature for aggregating performance metrics. These metrics may include various data related to organ transport logistics, such as the number of successful organ transports, the average time of organ transport, the cost of organ transport, and the satisfaction ratings of the transplant centers. The aggregation of these metrics may provide a comprehensive overview of the performance of the organ transport logistics, potentially helping to identify areas for improvement and optimize the process.

In other aspects, the mechanism may include a dashboard that displays the aggregated performance metrics in a user-friendly format. The dashboard may transform the raw data into interactive graphs and metrics, such as cost percentages and time estimates. This visual representation of the performance metrics may potentially make it easier for the users to understand and analyze the data, thereby potentially improving the decision-making process.

In some instances, the mechanism may include a feature for exporting the aggregated performance metrics. This feature may allow the users to download the metrics in various formats, such as CSV or PDF. The export feature may potentially facilitate the sharing and further analysis of the performance metrics, potentially contributing to the continuous improvement of organ transport logistics.

In some aspects, the digital SaaS solution may include a function for increasing transparency throughout the supply chain. This function may provide users with real-time visibility into the organ transport process, from the moment the organ is retrieved to the moment it is transplanted. The transparency may potentially improve the coordination of organ transport logistics, reduce uncertainties, and increase the confidence of the transplant centers.

In some cases, the function may include a feature for tracking the status of the organ transport in real-time. This feature may provide users with updates on various aspects of the organ transport, such as the location of the organ, the estimated time of arrival, and any changes in the transport plan. The real-time tracking may potentially allow users to make informed decisions and take timely actions, thereby potentially improving the efficiency and effectiveness of organ transport logistics.

In other aspects, the function may include a feature for providing users with access to historical transport data. This feature may compile and store data from past organ transports, such as the routes taken, the time taken, and the cost incurred. The historical transport data may potentially provide users with insights into the performance of the organ transport logistics, potentially helping to identify trends, benchmark performance, and inform future decision making.

In some instances, the function may include a feature for sharing information with all relevant parties. This feature may allow users to share updates, documents, and other information with all parties involved in the organ transport process, such as the transplant centers, the charter operators, and the PCC help desk staff. The sharing of information may potentially improve the collaboration and coordination among the parties, thereby potentially enhancing the overall transparency of the organ transport logistics.

In some aspects, the digital SaaS solution, the method, and the system may include a dashboard providing users with visual analytics and insights on completed organ transport trips. This dashboard may serve as a centralized hub for data visualization, potentially enabling users to gain a comprehensive understanding of the organ transport logistics. The dashboard may display various types of data, such as the number of completed trips, the average time of organ transport, the cost of organ transport, and the satisfaction ratings of the transplant centers. This visual representation of data may potentially facilitate the analysis and interpretation of the data, thereby potentially improving the decision-making process.

In some cases, the dashboard may transform historical transport data into interactive graphs and metrics. This transformation may involve the processing and analysis of raw data to generate meaningful insights. The interactive graphs and metrics may include various types of visualizations, such as bar charts, pie charts, line graphs, and scatter plots. These visualizations may represent various metrics, such as cost percentages and time estimates. The interactive nature of the graphs and metrics may allow users to manipulate the data, such as by adjusting the time period, selecting specific data points, and comparing different metrics. This interactive and visual representation of historical transport data may potentially make it easier for users to understand and analyze the data, thereby potentially contributing to the optimization of organ transport logistics.

In other aspects, the dashboard in the method and the system may provide users with visual analytics and insights on completed organ transport trips. This dashboard may serve as a tool for monitoring and managing the organ transport logistics. The visual analytics may include various types of data visualizations, such as heat maps, geographic maps, and trend lines. These visualizations may provide users with insights into various aspects of the organ transport logistics, such as the geographical distribution of organ transports, the trends in organ transport times, and the patterns in organ transport costs. The insights gained from these visual analytics may potentially inform the planning and execution of future organ transports, thereby potentially improving the efficiency and effectiveness of the method and the system.

In some instances, the dashboard may transform historical transport data into interactive graphs and metrics. This transformation may involve the use of data analysis techniques and visualization tools. The interactive graphs and metrics may provide users with a dynamic and intuitive way to explore and understand the historical transport data. The graphs may represent various aspects of the data, such as the distribution of organ transport times, the correlation between organ transport costs and distances, and the variation in organ transport times over different periods. The metrics may include various statistical measures, such as averages, medians, ranges, and percentiles. This transformation of historical transport data into interactive graphs and metrics may potentially enhance the usability and utility of the dashboard, thereby potentially contributing to the optimization of organ transport logistics.

In some aspects, the digital SaaS solution, the method, and the system may include a flight tracking feature that allows Transplant Centers to monitor shipments in real-time. This feature may provide users with real-time updates on the status and location of the organ transport. The real-time monitoring may potentially improve the coordination of organ transport logistics, reduce uncertainties, and increase the confidence of the transplant centers.

In some cases, the flight tracking feature may compile shipment routes over time. This compilation may involve the collection and storage of data on the routes taken by the organ transports. The historical tracking data may provide users with a record of past organ transports, potentially helping to identify trends, benchmark performance, and inform future decision making.

In other aspects, the flight tracking feature in the method and the system may allow Transplant Centers to monitor shipments in real-time. This feature may provide users with real time visibility into the organ transport process, potentially improving the coordination and efficiency of organ transport logistics.

In some instances, the flight tracking feature may compile shipment routes over time, building historical tracking data that Transplant Centers can analyze to inform future transportation preferences and decision making. This compilation may involve the processing and analysis of raw data to generate meaningful insights. The historical tracking data may provide users with a comprehensive overview of the performance of the organ transport logistics, potentially helping to identify areas for improvement and optimize the process.

In some aspects, the system may include a flight tracking feature for real-time monitoring of shipments. This feature may provide users with real-time updates on the status and location of the organ transport. The real-time monitoring may potentially improve the coordination of organ transport logistics, reduce uncertainties, and increase the confidence of the transplant centers.

In some cases, the flight tracking feature may compile shipment routes over time, building historical tracking data that Transplant Centers can analyze to inform future transportation preferences and decision making. This compilation may involve the collection and storage of data on the routes taken by the organ transports. The historical tracking data may provide users with a record of past organ transports, potentially helping to identify trends, benchmark performance, and inform future decision making.

In some aspects, the method for optimizing healthcare logistics may involve the creation and submission of Transport Requests. These requests may detail the transportation parameters for organ transport, such as the type and size of the organ, the distance and duration of the transport, and the urgency of the transport. The creation and submission of Transport Requests may be facilitated through the frontend user interface, which may provide a user-friendly interface for entering and submitting the transport details. This frontend user interface may potentially streamline the process of requesting organ transport, thereby potentially improving the efficiency of the method.

In some cases, the method may involve the receiving and reviewing of proposals from charter operators. These proposals may provide information on the availability and capabilities of the charter operators, such as the types of aircraft they operate, their flight schedules, and their pricing. The review of these proposals may involve the comparison and evaluation of the proposals based on various factors, such as the compatibility of the charter operators with the transport parameters, the reliability and reputation of the charter operators, and the cost effectiveness of their services. This review process may potentially ensure that the selected charter operator is suitable for the organ transport, thereby potentially improving the effectiveness of the method.

In other aspects, the method may involve the selection and confirmation of a preferred proposal. This selection may be based on the review of the proposals, and the confirmation may involve the sending of a confirmation message to the selected Charter Operator. The confirmation message may include details of the organ transport, such as the time and location of the pickup and delivery, the type and size of the organ, and any special handling instructions. This selection and confirmation process may potentially ensure that the organ transport is arranged in a timely and efficient manner, thereby potentially improving the efficiency and effectiveness of the method.

In some aspects, the digital SaaS solution, the method, and the system may involve the consolidation of charter details in a centralized hub. This consolidation may involve the collection, organization, and storage of data related to the charter operators, such as their availability, capabilities, flight schedules, and pricing. The centralized hub may provide a single point of access to these charter details, potentially simplifying the process of reviewing and selecting charter operators. This consolidation of charter details may potentially improve the efficiency and effectiveness of organ transport logistics.

In some cases, the centralized hub may include a feature for updating the charter details in real-time. This feature may allow the charter operators to update their details as they change, such as when they become available or unavailable, when they acquire new aircraft, or when they change their pricing. The real-time updating of charter details may potentially ensure that the information in the centralized hub is current and accurate, thereby potentially improving the reliability of the organ transport logistics.

In other aspects, the digital SaaS solution, the method, and the system may involve the provision of access to historical transport data and invoicing. This provision may involve the collection, organization, and storage of data related to past organ transports, such as the routes taken, the time taken, the cost incurred, and the satisfaction ratings of the transplant centers. The historical transport data may provide users with a record of past organ transports, potentially helping to identify trends, benchmark performance, and inform future decision making. The invoicing data may include details of the charges for the organ transport, such as the fees for the charter operators, the costs of fuel and maintenance, and any additional charges. The provision of access to this data may potentially improve the transparency and accountability of the organ transport logistics.

In some instances, the provision of access to historical transport data and invoicing may include a feature for exporting the data. This feature may allow users to download the data in various formats, such as CSV or PDF. The export feature may potentially facilitate the sharing and further analysis of the data, potentially contributing to the continuous improvement of organ transport logistics.

In some aspects, the system for streamlining organ transport logistics may include an integrated chat functionality for real-time communication. This functionality may enable secure direct messaging between all users, such as Transplant Coordinators, Charter Operators, and PCC help desk staff. The real-time communication may potentially improve the coordination of organ transport logistics, reduce uncertainties, and increase the confidence of the transplant centers.

In some cases, the system may include a dashboard providing visual analytics and insights on completed organ transport trips. This dashboard may transform historical transport data into interactive graphs and metrics, such as cost percentages and time estimates. The visual representation of data may potentially facilitate the analysis and interpretation of the data, thereby potentially improving the decision-making process.

In other aspects, the system may include a flight tracking feature for real-time monitoring of shipments. This feature may provide users with real-time updates on the status and location of the organ transport. The real-time monitoring may potentially improve the coordination of organ transport logistics, reduce uncertainties, and increase the confidence of the transplant centers.

In some instances, the flight tracking feature may compile shipment routes over time, building historical tracking data that Transplant Centers can analyze to inform future transportation preferences and decision making. This compilation may involve the collection and storage of data on the routes taken by the organ transport. The historical tracking data may provide users with a record of past organ transports, potentially helping to identify trends, benchmark performance, and inform future decision making.

In some aspects, the system may include a shipment reminder system for sending automated notifications. This system may automatically send notifications to relevant users at specified intervals throughout the organ transportation process. The automated notifications may potentially improve the coordination of organ transport logistics, reduce uncertainties, and increase the confidence of the transplant centers.

In some aspects, the digital SaaS solution, the method, and the system may include a central server hosted on AWS using a relational database (e.g., a Postgres database). This central server may serve as the foundation for the various functionalities and features of the solution, method, and system. The hosting on AWS may potentially provide scalability, reliability, and security, thereby potentially enhancing the performance and robustness of the solution, method, and system.

In some cases, the backend may include various components, such as servers, databases, and application programming interfaces (APis). These components may interact with each other to process requests, store data, and deliver responses. The use of AWS for hosting may potentially provide a scalable and flexible infrastructure, thereby potentially accommodating varying loads and demands.

In other aspects, the backend may use a relational database such as a Postgres database for storing and managing data. This database may include various tables and schemas for organizing the data, such as data on organ transport, charter operators, transplant centers, and users. The use of a relational database such as a Postgres database may potentially provide robust data management capabilities, thereby potentially ensuring the integrity and consistency of the data.

In some instances, the backend may include a feature for performing data backup and recovery. This feature may automatically backup the data in the relational database at regular intervals and recover the data in case of any data loss or corruption. The data backup and recovery feature may potentially enhance the reliability and resilience of the solution, method, and system.

In some aspects, the backend may include a feature for performing data analysis and reporting. This feature may process the data in the relational database to generate insights and reports, such as reports on organ transport performance, user activity, and system usage. The data analysis and reporting feature may potentially provide valuable insights for decision making and continuous improvement of the solution, method, and system.

A number of implementations have been described. Nevertheless, it will be understood that various modifications may be made without departing from the spirit and scope of the disclosure. Accordingly, other implementations are within the scope of the following claims.

For the purposes of this disclosure, the terms “organs,” “materials,” “perishables,” and “resources” may be used to refer to the subject objects of the systems and methods described herein. Accordingly, these terms may be used interchangeably throughout this disclosure to describe the items being transported, tracked, and managed by the transport logistics platform.

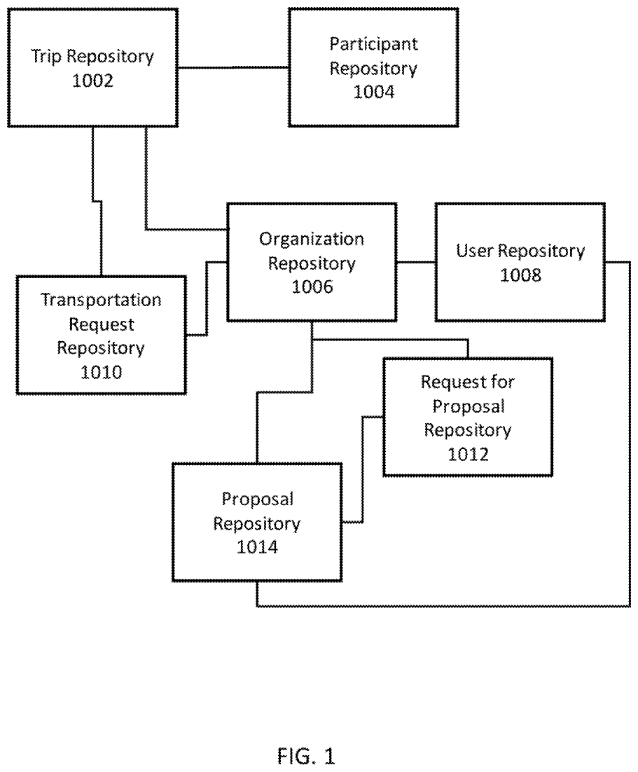

FIG. 1 is a database diagram for the digital SaaS solution for Critical Healthcare Logistics includes a platform for automating the process of organ transport logistics, a system for providing transplant centers with dedicated access to ground transportation and drivers as well as planes and pilots, a feature for enabling transplant coordinators to communicate directly with the relevant parties, a mechanism for streamlining invoicing and aggregating performance metrics, and a function for increasing transparency throughout the supply chain. The instant disclosure also envisions using both medical professionals and facilities at the transplant center as well as contract facilities, medical professionals and surgeons. The instant disclosure further anticipates using organ procurement organizations in addition to the resources at the transplant center.

The database diagram of FIG. 1 illustrates the data structure of the system of the present disclosure, comprising a plurality of interconnected repositories that store and manage data related to organ transport logistics operations. The trip repository 1002 may store data points including trip identifiers, current segment indicators, organ types, participant information, birthdate, charter names, charter identifiers, creation timestamps, delivery preferences, email addresses, gender, names, organization identifiers, phone numbers, user identifiers, weight information, city, county, country names, state codes, display names, formatted addresses, location coordinates including latitude and longitude, state, UTC offset minutes, zip codes, recipient facility information, recovery type, request routing parameters, scheduled departure and arrival times, status indicators, transportation identifiers, trip comments, channel identifiers, segment types, service times, tail numbers, and transport identifiers.

The participant repository 1004 may store data points including participant identifiers, birthdate, chat names, chat user identifiers, creation timestamps, status preferences, email addresses, first names, gender, identifiers, last names, organization identifiers, phone numbers, update timestamps, user identifiers, and weight in pounds.

The organization repository 1006 may store data points including organization identifiers, communication preferences, always add participant indicators, creation timestamps, feature flags, created audit information, description, enabled status, name, update timestamps, center email addresses, system enabled indicators, transportation facilities, city, county, country names, state codes, display names, drop-off times, formatted addresses, identifiers, location coordinates, longitude, latitude, preferred facilities, city, county, country names, state codes, display names, formatted addresses, identifiers, location coordinates, preferred facility indicators, safety preferences, transportation identifiers, air fleet information, insurance details, medical licensing, model specifications, power levels, special types, tail numbers, traveler capacities, vehicle identifiers, year information, support information, airport facility data, contact information, and version identifiers.

The user repository 1008 may store data points including user identifiers, added by user identifiers, added dates, creation timestamps, first names, identifiers, last login timestamps, last names, notification preferences, email addresses, image URLs, associated identifiers, user preferences, primary email addresses, primary phone numbers, role designations, secondary email addresses, secondary phone numbers, subscriber times, identifiers, labels, types, status indicators, titles, update timestamps, and verification email indicators.

The transportation request repository 1010 may store data points including request identifiers, version numbers, construction modes, creation timestamps, equipment return indicators, identifiers, air transportation preferences, expected travel parameters, organization identifiers, organ types, organ type identifiers, pickup location information including city, county, country names, state codes, display names, formatted addresses, identifiers, location coordinates, latitude, longitude, state, UTC offset minutes, zip codes, recipient facility information, pickup destination parameters, preferred facilities, city, county, country names, state codes, display names, formatted addresses, identifiers, location coordinates, recovery location information, recovery type indicators, special instructions, surgical service identifiers, transportation identifiers, air and ground indicators, ground options, transportation status indicators, trip status modes, order identifiers, and update timestamps.

The request for proposal repository 1012 may store data points including request for proposal identifiers, version numbers, accepted dates, acceptance decision indicators, creation timestamps, due dates, identifiers, request organization identifiers, transportation identifiers, and update timestamps.

The proposal repository 1014 may store data points including proposal identifiers, version numbers, acceptance indicators, additional information, administrator indicators, creation timestamps, markup percentages, previous landing information, price, proposal segment identifiers, air segment identifiers, air transportation identifiers, air service indicators, crew information, tail numbers, aircraft parameters, scheduled departure and arrival times, data coordinates, latitude, longitude, associated identifiers, distance metrics, scheduled information, proximity indicators, scheduled parameters, identifiers, data information, latitude, longitude, associated identifiers, distance metrics, scheduled information, proposal status indicators, request for proposal identifiers, source identifiers, transportation identifiers, and update timestamps.

The system may use matching algorithms implementing the matching criteria to match the requests to the ideal service providers, based on a variety of data points, including transplant center preferences, safety parameters, location, destinations, equipment, number of travelers, etc.

The system may use proprietary algorithms to optimize the route(s) recommended to recover organs. These recommendations may be based on the transplant center's preferences, origin and destination, distances, weather, traffic, transportation operator availability, etc.

The system may leverage modern cloud platforms that provide HIPAA compliant cloud services, modern communication mechanisms, and secure database technology in order to provide real-time data access and analytical insights to the users of the system as to the performance, efficiency, and effectiveness of the system. This new platform may replace current systems and methods that leverage manual methods of communication which delayed information delivery that expose the processes to risk due to miscommunication.

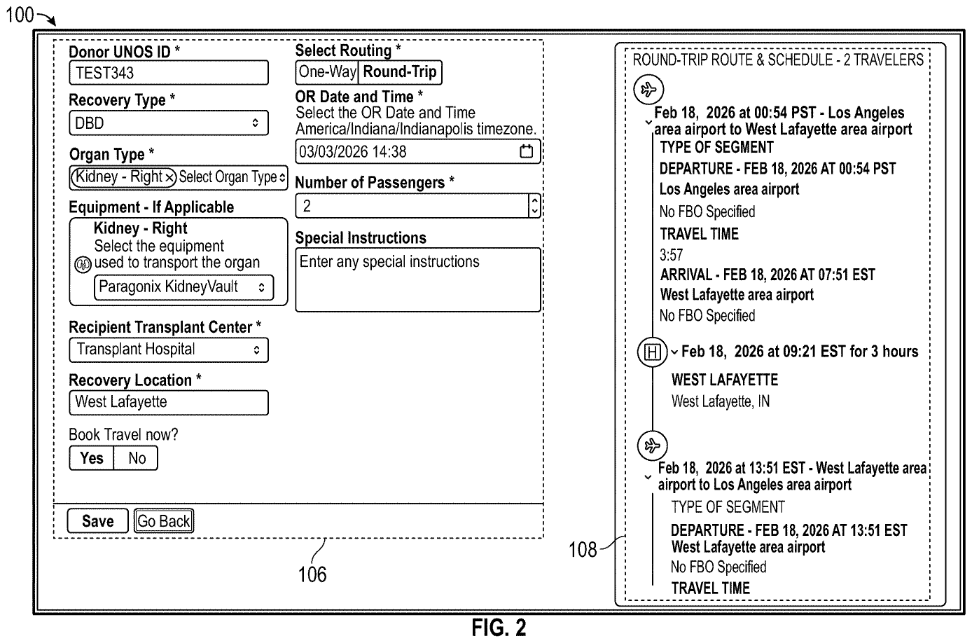

FIG. 2 is an illustration of a user interface screen 100 for a transportation request in accordance with one or more embodiments of the present disclosure. The user interface screen 100 may be rendered through the frontend user interface retrievable by users of the system, enabling transplant coordinators to create and submit transport requests detailing transportation requirements. The user interface screen 100 may include a transportation request user interface 106 configured to receive transport request inputs comprising organ type, recovery location, recipient facility, and timing requirements. The transportation request user interface 106 may display input fields for specifying a Donor UNOS ID, Recovery Type, Organ Type, and Equipment used to transport the organ, such as specialized preservation devices. The transportation request user interface 106 may further include fields for specifying the Recipient Transplant Center and Recovery Location, enabling the central server to process the transport request data and execute matching algorithms to identify appropriate transportation providers from the plurality of transportation providers stored in the relational database. The Select Routing section within the transportation request user interface 106 may provide options for One-Way and Round-Trip configurations, while the OR Date and Time field may capture timing requirements that the scheduling module uses to match transport requirements with available transportation resources based on factors comprising availability of aircraft, urgency of the organ transport, and distance between the recovery location and the recipient facility.

The user interface screen 100 may also include an itinerary timeline 108 positioned to display the sequenced transportation segments generated by the central server based on the routing optimization algorithms. The itinerary timeline 108 may present a visual representation of the Round-Trip Route and Schedule, displaying departure and arrival times, airport locations, and travel time details for each segment of the organ transport operation. The itinerary timeline 108 may show flight segments with departure and arrival information, including timezone specifications, enabling transplant coordinators to review the proposed route recommendations generated by the scheduling module that analyzes transplant center preferences, origin and destination locations, distances, weather data, traffic conditions, and transportation operator availability. The itinerary timeline 108 may further display ground segments with scheduled times and durations, illustrating the integration of multiple transportation modes coordinated by the central server. The information displayed in the itinerary timeline 108 may correspond to the itinerary data structure generated by the central server and stored in the relational database, which comprises sequenced transportation segments, timing parameters, location coordinates, and participant assignments that are transmitted to electronic devices associated with all designated participants.

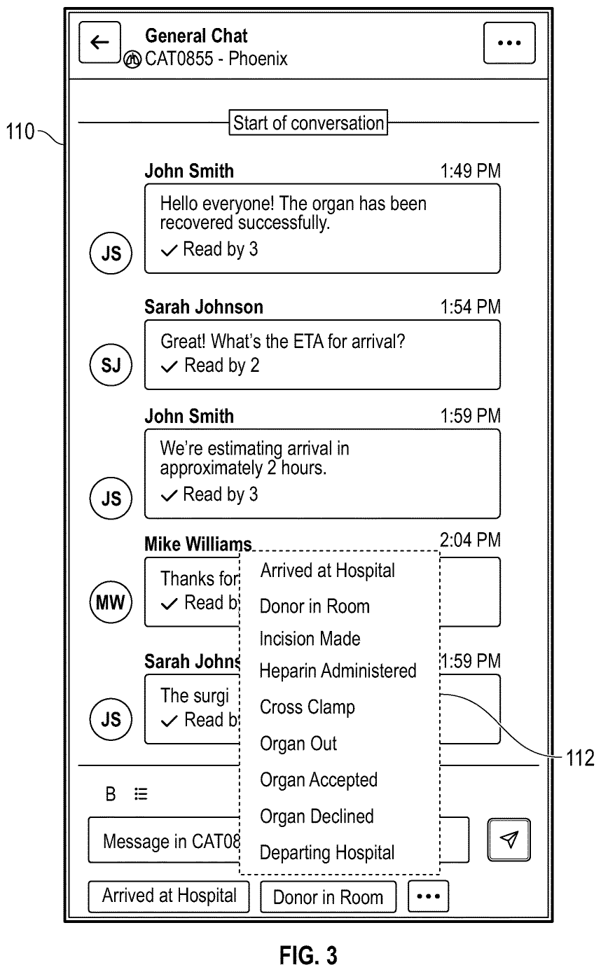

FIG. 3 is an illustration of a UI screen for a messaging feature of the system in accordance with one or more embodiments of the present disclosure. The figure depicts a messaging interface 110 that implements the integrated chat functionality enabling secure direct messaging between transplant coordinators, charter operators, drivers, pilots, and help desk staff in real-time during organ transport operations. The messaging interface 110 may be rendered through the frontend user interface and configured to facilitate communication through the chat module of the central server. The messaging interface 110 may display a conversation thread associated with a specific case identifier, showing messages exchanged between multiple users involved in the organ transport chain. The messaging interface 110 may include message bubbles with timestamps and read receipt indicators, enabling users to track communication status and ensure that time-sensitive information regarding organ assignments and transport issues has been received by relevant participants. The chat module may process and store communication records in the relational database, maintaining a complete record of all messages exchanged during the organ transport operation for compliance and audit purposes.

The messaging interface 110 may further include a monitoring dashboard 112 configured to enable users to communicate standardized clinical timestamps during the organ procurement procedure. The monitoring dashboard 112 may be displayed as a dropdown menu overlay presenting selectable clinical milestone options comprising Arrived at Hospital, Donor in Room, Incision Made, Heparin Administered, Cross Clamp, Organ Out, Organ Accepted, Organ Declined, and Departing Hospital. The monitoring dashboard 112 may enable the central server to receive clinical timestamp data from electronic devices associated with the transplant recovery team or donor facility contacts, wherein the clinical timestamp data comprises procedure milestone indicators, timestamp values, and organ status information corresponding to events occurring during the organ procurement procedure. The central server may process the received clinical timestamp data in real-time and transmit status update notifications to all designated participants, enabling coordinated preparation for subsequent transportation segments. Quick-access buttons displayed at the bottom of the messaging interface 110 may allow users to send predefined status updates through the monitoring dashboard 112, facilitating rapid communication of recovery initiation signals and recovery completion signals that trigger dispatch activation signals to ground transportation providers and flight initiation signals to air transportation crew.

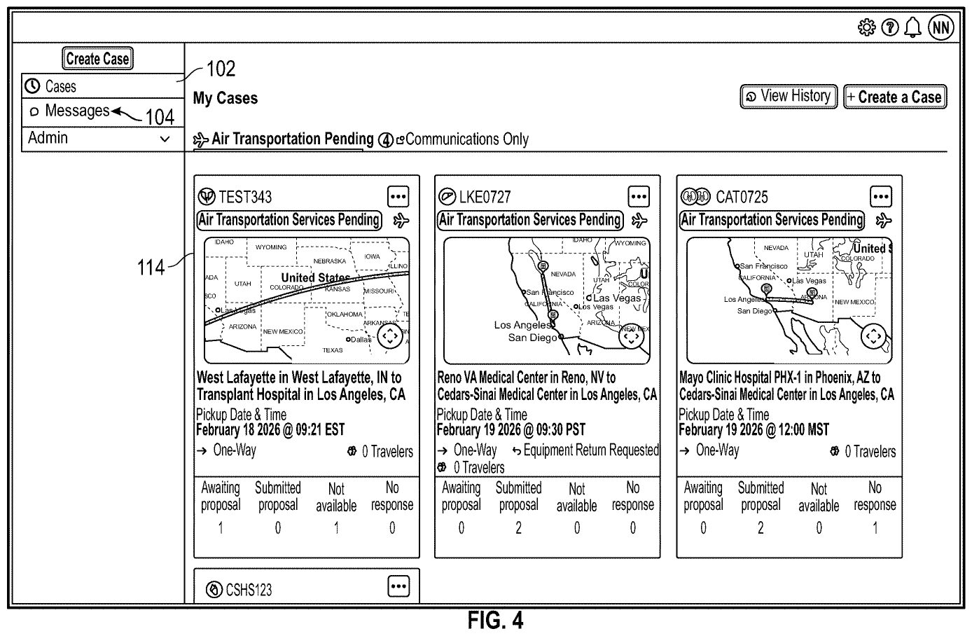

FIG. 4 is an illustration of a UI screen for a pending transportation request in accordance with one or more embodiments of the present disclosure. The figure depicts a case management dashboard rendered through the frontend user interface, enabling transplant coordinators to view and manage multiple organ transport cases simultaneously. The navigation panel on the left side of the screen may include a transportation panel 102 labeled as Cases and a communication panel 104 labeled as Messages, providing access to the various functionalities of the digital SaaS solution. The transportation panel 102 may enable users to access transport request data stored in the relational database, while the communication panel 104 may provide access to the messaging interface 110 and the integrated chat functionality for real-time communication with participants in the organ transport chain. The case management dashboard may consolidate charter details in a centralized hub, providing a single point of access to transport request information, proposal status, and route details for each pending organ transport operation.

The main content area of the UI screen may display a case interface 114 showing multiple pending transportation cases arranged in a card-based layout. Each case card within the case interface 114 may display a case identifier, status indicator, and map visualization depicting the transport route between origin and destination locations generated by the routing optimization algorithms. The case interface 114 may present proposal status information showing counts for proposals in various states including Awaiting proposal, Submitted proposal, Not available, and No response categories, enabling transplant coordinators to track the progress of the request for proposal process executed by the central server. The case cards may display pickup date and time information, trip type indicators, traveler count, and equipment return request status, corresponding to the transport request data received by the central server from the frontend user interface. The case interface 114 may enable transplant coordinators to review submitted proposals and select to accept, request update, or reject proposals from the aggregated proposal data transmitted by the central server, facilitating the selection and confirmation of preferred proposals from multiple charter operators and ground transportation providers matched by the proprietary matching algorithms.

The features depicted in FIGS. 2-4, including the user interface screen 100, the transportation request user interface 106, the itinerary timeline 108, the messaging interface 110, the monitoring dashboard 112, the transportation panel 102, the communication panel 104, and the case interface 114, may be automatically updated based on communication between the central server and the various electronic devices assigned to members of the organ transport chain implicated in a given transportation operation. The central server may continuously receive data transmissions from electronic devices associated with transplant coordinators, charter operators, air transportation crew, ground transportation drivers, donor facility contacts, and transplant recovery team members, processing the received data to update the displayed information in real-time across all user interface elements. Through this automated communication architecture, the user interface screen 100 may provide a real-time and highly accurate understanding of the progress of a given actor within the organ transport chain, reflecting current location data, completed milestones, and active status indicators as they occur. The user interface screen 100 may further display the availability of a given actor, enabling transplant coordinators to identify which transportation providers, pilots, drivers, and medical personnel are currently accessible and ready to participate in pending or active transport operations. Additionally, the user interface screen 100 may present the capacity of a given actor, including available aircraft specifications, vehicle configurations, crew assignments, and scheduling constraints that inform the matching algorithms and route optimization processes executed by the central server. This continuous synchronization between the central server and the distributed electronic devices throughout the organ transport chain may ensure that all participants have access to current and accurate information, potentially reducing delays and miscommunication during time-critical organ transport operations.

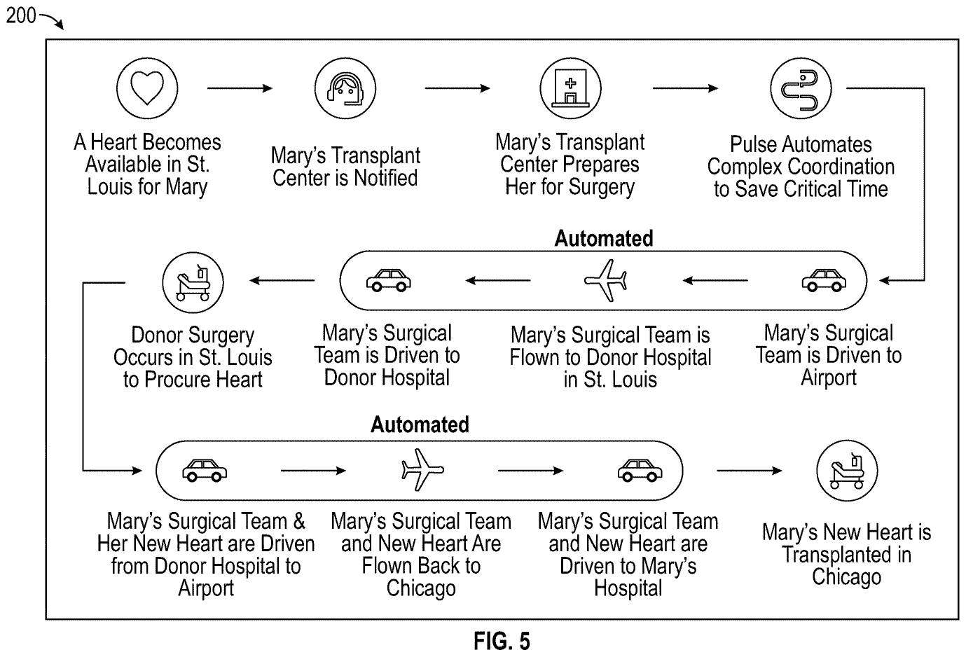

FIG. 5 is an illustration of an example use-case scenario 200 in accordance with one or more embodiments of the present disclosure. The use-case scenario 200 may depict a complete workflow for organ transport logistics, illustrating the sequence of steps and coordination involved in transporting a heart from a donor location in St. Louis to a recipient location in Chicago. The use-case scenario 200 may demonstrate how the system automates complex coordination to save critical time during the organ transport process, beginning when an organ becomes available and the Transplant Center is notified. The workflow may proceed through multiple transportation segments including ground transportation of the surgical team to an airport, air transportation to the donor hospital location, ground transportation to the donor facility, organ recovery surgery, and the return journey with the procured organ. The use-case scenario 200 may illustrate the integration of multiple transportation modes, including ground transportation and air transportation, demonstrating how the system may coordinate both automated ground and air transportation segments to facilitate the timely delivery of organs from donor facilities to recipient facilities. This use-case scenario 200 may highlight the platform's capability for providing transplant centers with dedicated access to planes and pilots as well as ground transportation and drivers, while potentially enabling Transplant Coordinators to communicate directly with the relevant parties throughout the entire process.

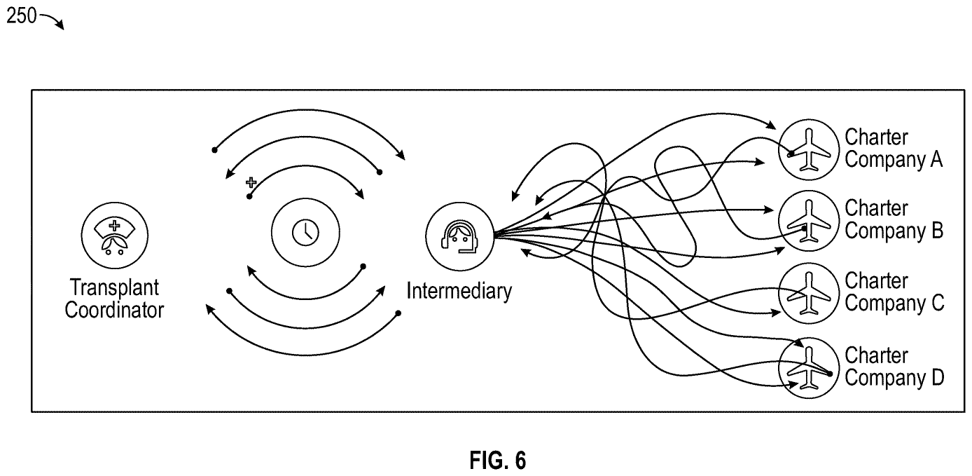

FIG. 6 is an illustration of a communication structure 250 in accordance with one or more embodiments of the present disclosure. The communication structure 250 may depict the flow of information and coordination between participants in the organ transport logistics system, illustrating how the digital SaaS solution may facilitate communication between Transplant Coordinators and multiple Charter Companies through a centralized intermediary. On the left side of the communication structure 250, a Transplant Coordinator may be represented, communicating with an Intermediary positioned in the center through bidirectional arrows indicating the exchange of information in both directions. The Intermediary may serve as a central coordination point within the communication structure 250, potentially facilitating the connection between the Transplant Coordinator and multiple transportation service providers including Charter Company A, Charter Company B, Charter Company C, and Charter Company D. The multiple connection lines extending from the Intermediary to the Charter Companies may illustrate the simultaneous communication capability of the system, potentially enabling the transmission of transport requests and receipt of proposals from multiple charter operators based on the proprietary matching algorithms. This configuration within the communication structure 250 may represent the platform's ability to automate the process of organ transport logistics by providing transplant centers with access to a network of transportation providers, potentially streamlining the request for proposal process and enabling the review and selection of preferred proposals from multiple service providers.

In some aspects, each actor within the organ transport chain, including pilots, drivers, surgeons, perfusionists, transplant coordinators, and other healthcare professionals, may be in possession of or in proximity to an electronic device with GPS capabilities. Such electronic devices, which may include smartphones, tablets, or dedicated tracking devices, may execute a user-end version of the instant application or may provide location data to the central server via alternative communication protocols such as cellular data transmission, Wi-Fi connectivity, or satellite communication. Through this configuration, the electronic devices may continuously or periodically transmit GPS coordinates to the central server, which may store the location data in the relational database and process the data through the tracking module. By associating a given electronic device with a specific transport operation or a specific organ through participant designation data stored in the relational database, the organ may be tracked by virtue of tracking the location of the actor's electronic device. This association may enable the central server to determine the real-time location of an organ throughout the transport chain by monitoring the GPS coordinates transmitted from the electronic device of the actor currently in possession of or responsible for the organ.

In some cases, when a given actor is utilizing a vehicle such as an automobile, truck, or helicopter, the onboard electronics of that vehicle may be configured to transmit GPS location data to the central server. Vehicle telematics systems, onboard navigation units, or integrated fleet management devices may ping GPS coordinates at regular intervals or upon request from the central server. The central server may receive this vehicle-based location data through dedicated communication channels or through integration with fleet management platforms, storing the received coordinates in the relational database and associating the vehicle location with the corresponding transport operation. This vehicle-based tracking may provide enhanced accuracy and reliability compared to personal electronic devices, particularly during extended ground transportation segments.

In other aspects, third-party application programming interfaces such as flight tracking programs, aviation data services, and air traffic monitoring systems may be integrated with the central server to enable location determination for aircraft during air transportation segments. The central server may interface with these external APIs to retrieve real-time flight position data, altitude information, speed metrics, and estimated arrival times for aircraft involved in organ transport operations. This integration may enable the tracking module to monitor the progress of air transportation segments without requiring direct communication with the aircraft or the electronic devices of passengers aboard the aircraft.

In some instances, the central server may be configured to seamlessly transition between tracking schemas based on the current phase of the transport operation. For example, the central server may track the electronic device of the pilot or the party carrying the organ during ground-based movement leading up to boarding an aircraft, but may automatically switch to retrieving location data from the flight tracking API when the aircraft takes off and cellular GPS signals from personal electronic devices would otherwise be unavailable or unreliable. This transition may be triggered by the appearance of movement data or signal acquisition from the secondary tracking source, such as the flight tracking API detecting aircraft departure, or may alternatively be triggered by discontinuation of GPS pings or data transmissions from the primary tracking source, such as the electronic device indicating that the device has entered an environment where cellular communication is restricted. Through this adaptive tracking architecture, the central server may maintain continuous and uninterrupted location monitoring throughout the entire organ transport operation, ensuring that transplant coordinators and other designated participants have access to accurate real-time location information regardless of the transportation mode or communication constraints encountered during transit.

The system may manage the process through multiple phases:

Request for transportation of organ recovery—The ability for authorized users to make a request to a market of service providers to recover organ(s) and deliver them to the recipient for transplantation. During the request process, the system may make recommendations for routing based on routing optimization algorithms mentioned above.

Proposal—The system may match service providers to the request based on the matching algorithms implementing the matching criteria mentioned above. The system may allow matched service providers to submit their proposals to supply the applicable services, including surgical services, air, helicopter, or ground transportation services. The system will allow the transplant centers to review and respond to the multiple proposals.

Organ Recovery—The system may manage the organ recovery process providing transparency and communication of current status during the full organ recovery process. All parties involved, including transplant center, organ recovery teams, donor facility teams, service providers, etc., may have real-time access to the current status of the organ recovery and be able to communicate securely with other relevant participants. During the organ recovery process, the system may continually monitor current progress and may leverage the routing optimization algorithms mentioned above to optimize the process to increase organ recovery rates.

The following is one example method for receiving and managing transportation requests in accordance with one or more embodiments of the present disclosure:
Organ is accepted by transplant center.
Transplant center logs into the PCC portal to request transportation for both ground and air.
Transportation request first goes out to select air transportation operators for a request for proposal (hereinafter “RFP”) based on matching algorithms.
Matched air transportation operators are notified of pending RFP's.
Air transportation operator accepts or declines to participate in RFP.
Air transportation operators participating in RFP are notified of pending submission of proposal until proposal is submitted.

Air transportation operator may create a proposal with price and details of the trip.
Transplant center reviews submitted proposals and can reject, request update, or accept a proposal.
Air transportation operators are notified of transplant center's decision.
After air charter proposal accepted, proposal goes out to ground transportation operators at recipient and donor locations (could be many or could be preferred vendor) based on matching algorithms.
For certain geographies, need to configure both helicopters and ground, dependencies on weather.
Ground transportation operators accept or decline to participate in RFP.
Ground transportation operators are notified of RFP's that are pending submission of proposal until the proposal is submitted.
Ground transportation creates proposal with price and details of the trip.
Transplant center is notified of submitted proposals.
Transplant center reviews submitted proposals and can accept, request updates or reject proposals.
Ground transportation operators are notified of transplant center's decision.
Once ground transportation is accepted, the organ transplant process moves into organ recovery.

In some aspects, the present disclosure relates to a method 300 for receiving and managing transportation requests in accordance with one or more embodiments. The method 300 may be executed by a central server in communication with a plurality of electronic devices associated with various actors in the organ transport logistics chain.

At step 302, the method 300 may comprise receiving, by the central server, an organ acceptance signal from a first electronic device associated with a transplant center. The organ acceptance signal may comprise data indicating that the transplant center has accepted an organ for transplantation. In some embodiments, this step corresponds to the organ being accepted by the transplant center, wherein the central server receives and stores acceptance data in the relational database, the acceptance data comprising organ identification information, donor information, and recipient information.

At step 304, the method 300 may comprise receiving, by the central server, transport request data from a user interface of the first electronic device. The transport request data may comprise parameters defining transportation requirements for the organ transport operation. In some embodiments, this step corresponds to the transplant center logging into the PCC portal to request transportation for both ground and air, wherein the central server receives authentication credentials, validates user authorization, and receives transport request inputs comprising origin location, destination location, timing requirements, and transportation mode specifications.

At step 306, the method 300 may comprise executing, by the central server, a matching algorithm to identify a subset of transportation providers from a plurality of transportation providers stored in the relational database. The matching algorithm may analyze the transport request data against transportation provider capability data to generate a matched provider list. In some embodiments, this step corresponds to the transportation request first going out to select air transportation operators for a request for proposal based on matching algorithms, wherein the central server processes the transport request data through proprietary matching algorithms that evaluate data points comprising transplant center preferences, safety requirements, location proximity, destination accessibility, equipment specifications, and traveler capacity.

At step 308, the method 300 may comprise transmitting, by the central server, notification signals to electronic devices associated with the matched transportation providers. The notification signals may comprise data indicating the existence of a pending request for proposal. In some embodiments, this step corresponds to matched air transportation operators being notified of pending RFPs, wherein the central server generates and transmits electronic notifications via the communication subsystem to operator devices, the notifications comprising request identifiers and summary information.

At step 310, the method 300 may comprise receiving, by the central server, participation response data from the electronic devices associated with the matched transportation providers. The participation response data may comprise acceptance or declination indicators. In some embodiments, this step corresponds to the air transportation operator accepting or declining to participate in the RFP, wherein the central server receives binary response signals, updates participation status records in the relational database, and adjusts the active provider list accordingly.

At step 312, the method 300 may comprise transmitting, by the central server, reminder notification signals to electronic devices associated with participating transportation providers at specified intervals. The reminder notification signals may indicate pending proposal submission requirements. In some embodiments, this step corresponds to air transportation operators participating in the RFP being notified of pending submission of proposal until the proposal is submitted, wherein the central server monitors proposal submission status and generates automated reminder transmissions based on configurable time intervals stored in the database.

At step 314, the method 300 may comprise receiving, by the central server, proposal data from electronic devices associated with the participating transportation providers. The proposal data may comprise pricing information, service specifications, and trip details. In some embodiments, this step corresponds to the air transportation operator creating a proposal with price and details of the trip, wherein the central server receives structured proposal data comprising cost figures, aircraft specifications, crew information, estimated departure and arrival times, and route information, and stores the proposal data in the relational database.

At step 316, the method 300 may comprise aggregating, by the central server, the received proposal data and transmitting the aggregated proposal data to the first electronic device associated with the transplant center. The aggregated proposal data may be formatted for display through the user interface. In some embodiments, this step corresponds to the transplant center reviewing submitted proposals and being able to reject, request update, or accept a proposal, wherein the central server compiles proposal records, generates comparison metrics, and transmits the aggregated data for rendering in the frontend user interface.

At step 318, the method 300 may comprise receiving, by the central server, a selection signal from the first electronic device indicating a decision regarding the proposals. The selection signal may comprise acceptance, rejection, or modification request indicators. In some embodiments, this step corresponds to the transplant center reviewing submitted proposals and selecting to reject, request update, or accept a proposal, wherein the central server receives the selection input, updates proposal status records in the relational database, and triggers subsequent workflow processes based on the selection type.

At step 320, the method 300 may comprise transmitting, by the central server, decision notification signals to electronic devices associated with the transportation providers. The decision notification signals may comprise data indicating the transplant center's decision regarding each proposal. In some embodiments, this step corresponds to air transportation operators being notified of the transplant center's decision, wherein the central server generates and transmits notification signals comprising acceptance confirmation, rejection notification, or modification request details to the respective operator devices.

At step 322, the method 300 may comprise executing, by the central server, a secondary matching algorithm to identify ground transportation providers upon acceptance of an air charter proposal. The secondary matching algorithm may analyze location data for both recipient and donor facilities. In some embodiments, this step corresponds to the proposal going out to ground transportation operators at recipient and donor locations based on matching algorithms after the air charter proposal is accepted, wherein the central server processes geographic data, preferred vendor lists, and ground transportation provider capability data to generate matched ground provider lists for multiple locations.

At step 324, the method 300 may comprise analyzing, by the central server, geographic and environmental data to determine appropriate transportation mode configurations. The analysis may evaluate weather data, terrain data, and distance data to generate transportation mode recommendations. In some embodiments, this step corresponds to configuring both helicopters and ground for certain geographies with dependencies on weather, wherein the central server retrieves weather forecast data from external data sources, analyzes geographic constraints, and determines appropriate transportation mode combinations based on the analyzed data. The central server may interface with application programming interfaces of weather service providers to retrieve current conditions, forecasts, and severe weather alerts that could impact air or ground transportation safety and timing along potential routes. Geographic information system data and mapping APIs may be accessed to evaluate terrain characteristics, road conditions, airport accessibility, and helipad availability at donor and recipient facilities. Real-time transportation models may integrate traffic data, flight tracking information, and historical performance metrics to generate dynamic recommendations that account for current operational conditions and optimize the selection between aircraft, helicopter, and ground vehicle configurations. The central server may communicate with these transportation models via an application programming interface (API) to retrieve and process the relevant data for route optimization and transportation mode selection.

At step 326, the method 300 may comprise receiving, by the central server, ground transportation participation response data from electronic devices associated with matched ground transportation providers. The participation response data may comprise acceptance or declination indicators. In some embodiments, this step corresponds to ground transportation operators accepting or declining to participate in the RFP, wherein the central server receives response signals, updates participation records, and maintains active ground provider lists in the relational database.

At step 328, the method 300 may comprise transmitting, by the central server, reminder notification signals to electronic devices associated with participating ground transportation providers. The reminder notification signals may indicate pending proposal submission requirements. In some embodiments, this step corresponds to ground transportation operators being notified of RFPs that are pending submission of proposal until the proposal is submitted, wherein the central server monitors ground proposal submission status and generates automated reminder transmissions.

At step 330, the method 300 may comprise receiving, by the central server, ground transportation proposal data from electronic devices associated with participating ground transportation providers. The ground transportation proposal data may comprise pricing information and trip specifications. In some embodiments, this step corresponds to ground transportation creating a proposal with price and details of the trip, wherein the central server receives structured proposal data comprising cost figures, vehicle specifications, driver information, and estimated pickup and delivery times.

At step 332, the method 300 may comprise transmitting, by the central server, notification signals to the first electronic device indicating receipt of ground transportation proposals. The notification signals may trigger user interface updates for proposal review. In some embodiments, this step corresponds to the transplant center being notified of submitted proposals, wherein the central server generates notification transmissions and updates the frontend user interface to display the newly received ground transportation proposals.

At step 334, the method 300 may comprise receiving, by the central server, ground transportation selection signals from the first electronic device. The selection signals may comprise acceptance, rejection, or modification request indicators for the ground transportation proposals. In some embodiments, this step corresponds to the transplant center reviewing submitted proposals and being able to accept, request updates, or reject proposals, wherein the central server receives selection inputs, updates ground proposal status records, and processes the selection decisions.

At step 336, the method 300 may comprise transmitting, by the central server, decision notification signals to electronic devices associated with the ground transportation providers. The decision notification signals may comprise data indicating the transplant center's decision. In some embodiments, this step corresponds to ground transportation operators being notified of the transplant center's decision, wherein the central server generates and transmits notification signals to ground operator devices.

At step 338, the method 300 may comprise transitioning, by the central server, the transport request status to an organ recovery phase upon acceptance of ground transportation proposals. The transition may trigger initialization of tracking and communication modules. In some embodiments, this step corresponds to the organ transplant process moving into organ recovery once ground transportation is accepted, wherein the central server updates the transport request status in the relational database, initializes real-time tracking data collection, and activates communication channels for all designated participants.

The following is one example method for transporting materials and organ recovery in accordance with one or more embodiments of the present disclosure:
Participants are designated (for communication and notification purposes):
   Transplant coordinator(s);
   Transplant recovery team (surgeons, perfusionists, possibly coordinator);
   Donor facility contacts;
   Air transportation coordinator contacts;
   Air transportation crew (pilots, other crew and tail numbers);
   Ground transportation coordinator contacts; and
   Ground transportation crew (drivers and vehicle identification).
Itinerary is finalized and communicated to all participants.
Ground transportation picks up transplant recovery team at recipient facility and transports to origin airport.
Air transportation delivers transplant recovery team to destination airport.
Ground transportation picks up transplant recovery team at destination airport and drives them to the donor facility and waits at hospital for organ recovery.
Recovery begins.
After recovery, ground transportation drives transplant recovery team to destination airport.
Air transportation delivers transplant recovery team and organ to origin airport.
Ground transportation picks up transplant recovery team at origin airport and drives them to recipient facility.
Organ recovery is complete.

In some aspects, the present disclosure relates to a method 400 for transporting materials and organ recovery in accordance with one or more embodiments. The method 400 may be executed by a central server in communication with a plurality of electronic devices associated with various participants in the organ transport logistics chain.

At step 402, the method 400 may comprise receiving, by the central server, participant designation data from electronic devices associated with authorized users. The participant designation data may comprise identification information, role classifications, and communication endpoint data for each participant involved in the transport operation. In some embodiments, this step corresponds to participants being designated for communication and notification purposes, wherein the central server receives and stores designation records comprising transplant coordinator identifiers, transplant recovery team member data including surgeons, perfusionists, and coordinators, donor facility contact information, air transportation coordinator contact data, air transportation crew information including pilots, other crew members, and aircraft tail numbers, ground transportation coordinator contact data, and ground transportation crew information including driver identifications and vehicle identification numbers.

At step 404, the method 400 may comprise generating, by the central server, an itinerary data structure based on the accepted proposals and participant designation data. The itinerary data structure may comprise sequenced transportation segments, timing parameters, location coordinates, and participant assignments. The central server may transmit the finalized itinerary data to electronic devices associated with all designated participants. In some embodiments, this step corresponds to the itinerary being finalized and communicated to all participants, wherein the central server compiles route information, departure and arrival times, pickup and delivery locations, and participant responsibilities into a structured itinerary record, stores the itinerary in the relational database, and transmits notification signals containing the itinerary data to each participant's electronic device.

At step 406, the method 400 may comprise transmitting, by the central server, dispatch signals to electronic devices associated with ground transportation providers at the recipient facility location. The dispatch signals may comprise pickup location coordinates, passenger manifest data, and destination airport information. The central server may receive confirmation signals and real-time location data from the ground transportation provider's electronic device during transit. In some embodiments, this step corresponds to ground transportation picking up the transplant recovery team at the recipient facility and transporting them to the origin airport, wherein the central server monitors GPS data streams from the ground vehicle, updates transport status records in the relational database, and transmits progress notifications to relevant participants.

At step 408, the method 400 may comprise transmitting, by the central server, flight initiation signals to electronic devices associated with air transportation crew upon confirmation of passenger arrival at the origin airport. The flight initiation signals may comprise passenger manifest data, destination airport identifiers, and flight plan parameters. The central server may receive flight tracking data from aviation data sources and aircraft transponder systems during the flight. In some embodiments, this step corresponds to air transportation delivering the transplant recovery team to the destination airport, wherein the central server monitors flight progress through integration with flight tracking APIs, updates transport status records, and transmits estimated arrival notifications to ground transportation providers and donor facility contacts.

At step 410, the method 400 may comprise transmitting, by the central server, secondary dispatch signals to electronic devices associated with ground transportation providers at the destination airport location. The secondary dispatch signals may comprise pickup location coordinates at the destination airport, passenger manifest data, and donor facility address information. The central server may receive confirmation signals indicating passenger pickup and real-time location data during transit to the donor facility. In some embodiments, this step corresponds to ground transportation picking up the transplant recovery team at the destination airport and driving them to the donor facility, wherein the central server monitors vehicle location data, calculates estimated arrival times at the donor facility, and transmits arrival notifications to donor facility contacts.

At step 412, the method 400 may comprise receiving, by the central server, standby status signals from electronic devices associated with ground transportation providers at the donor facility. The standby status signals may comprise vehicle location confirmation data and driver availability indicators. The central server may maintain active communication channels with the ground transportation provider during the standby period. In some embodiments, this step corresponds to ground transportation waiting at the hospital for organ recovery, wherein the central server logs standby initiation timestamps, monitors driver status through periodic check-in signals, and maintains readiness state records in the relational database.

At step 414, the method 400 may comprise receiving, by the central server, recovery initiation signals from electronic devices associated with the transplant recovery team or donor facility contacts. The recovery initiation signals may comprise timestamp data indicating commencement of the organ procurement procedure. The central server may update transport status records and transmit notification signals to all designated participants. In some embodiments, this step corresponds to recovery beginning, wherein the central server logs the recovery start timestamp, updates the transport request status to an active recovery phase, and transmits status update notifications to transplant coordinators, air transportation crew, and ground transportation providers.

At step 416, the method 400 may comprise receiving, by the central server, recovery completion signals from electronic devices associated with the transplant recovery team. The recovery completion signals may comprise timestamp data, organ condition data, and departure readiness indicators. The central server may transmit dispatch activation signals to the ground transportation provider's electronic device. In some embodiments, this step corresponds to recovery completion and ground transportation driving the transplant recovery team to the destination airport after recovery, wherein the central server logs recovery completion timestamps, updates organ status records, monitors vehicle location data during transit to the destination airport, and transmits estimated departure notifications to air transportation crew.

At step 418, the method 400 may comprise transmitting, by the central server, return flight initiation signals to electronic devices associated with air transportation crew upon confirmation of passenger and organ arrival at the destination airport. The return flight initiation signals may comprise updated passenger manifest data including organ transport indicators, origin airport identifiers, and return flight plan parameters. The central server may receive flight tracking data during the return flight and monitor organ transport conditions. In some embodiments, this step corresponds to air transportation delivering the transplant recovery team and organ to the origin airport, wherein the central server monitors return flight progress through flight tracking API integration, updates transport status records with organ-in-transit indicators, and transmits estimated arrival notifications to ground transportation providers at the origin airport and recipient facility contacts.

At step 420, the method 400 may comprise transmitting, by the central server, final dispatch signals to electronic devices associated with ground transportation providers at the origin airport location. The final dispatch signals may comprise pickup location coordinates at the origin airport, passenger and organ manifest data, and recipient facility address information. The central server may receive confirmation signals and real-time location data during the final transit segment. In some embodiments, this step corresponds to ground transportation picking up the transplant recovery team at the origin airport and driving them to the recipient facility, wherein the central server monitors vehicle location data, calculates estimated arrival times at the recipient facility, transmits arrival notifications to transplant coordinators and surgical preparation teams, and updates transport status records.

At step 422, the method 400 may comprise receiving, by the central server, delivery confirmation signals from electronic devices associated with ground transportation providers or recipient facility contacts. The delivery confirmation signals may comprise timestamp data indicating arrival of the transplant recovery team and organ at the recipient facility. The central server may update the transport request status to a completed state and generate completion records. In some embodiments, this step corresponds to organ recovery being complete, wherein the central server logs delivery completion timestamps, updates the transport request status in the relational database to indicate successful completion, generates performance metric data for the completed transport operation, and transmits completion notifications to all designated participants.

The following is one example method for communication between a transplant center team and an internal team for the transportation of materials in accordance with one or more embodiments of the present disclosure:
Ground will notify the transplant center of the estimated time of arrival, when they are there, etc.
Air will share itinerary.
Clinical timestamps will be shared as they occur at procurement OR.
ETA for the way back will also be shared.

In some aspects, the present disclosure relates to a method 500 for communication between a transplant center team and an internal team for the transportation of materials in accordance with one or more embodiments. The method 500 may be executed by a central server in communication with a plurality of electronic devices associated with various participants in the organ transport logistics chain.

At step 502, the method 500 may comprise receiving, by the central server, estimated time of arrival data from electronic devices associated with ground transportation providers. The estimated time of arrival data may comprise current vehicle location coordinates, calculated travel time based on route analysis, and traffic condition indicators. The central server may process the received location data through routing optimization algorithms to generate refined arrival estimates. In some embodiments, this step corresponds to ground notifying the transplant center of the estimated time of arrival, wherein the central server receives GPS data streams from ground transportation vehicles, analyzes the data against stored route information and real-time traffic data retrieved from external data sources, calculates updated arrival estimates, and transmits notification signals containing the estimated time of arrival to electronic devices associated with transplant coordinators at the transplant center.

At step 504, the method 500 may comprise receiving, by the central server, arrival confirmation signals from electronic devices associated with ground transportation providers. The arrival confirmation signals may comprise timestamp data indicating arrival at designated locations, vehicle location confirmation data, and status indicators. The central server may update transport status records in the relational database and generate notification transmissions to relevant participants. In some embodiments, this step corresponds to ground notifying the transplant center when they are there, wherein the central server logs arrival timestamps, updates the transport request status to reflect current progress, and transmits arrival notification signals to electronic devices associated with transplant coordinators, surgical preparation teams, and other designated participants.

At step 506, the method 500 may comprise generating, by the central server, itinerary data structures based on flight plan parameters received from electronic devices associated with air transportation crew. The itinerary data structures may comprise departure times, arrival times, airport identifiers, aircraft tail numbers, and crew information. The central server may transmit the itinerary data to electronic devices associated with all designated participants in the organ transport chain. In some embodiments, this step corresponds to air sharing the itinerary, wherein the central server compiles flight schedule information, route data, and crew assignments into structured itinerary records, stores the itinerary data in the relational database, and transmits notification signals containing the itinerary information to electronic devices associated with transplant coordinators, ground transportation providers at origin and destination airports, and donor facility contacts.

At step 508, the method 500 may comprise receiving, by the central server, clinical timestamp data from electronic devices associated with the transplant recovery team or donor facility contacts. The clinical timestamp data may comprise procedure milestone indicators, timestamp values, and organ status information corresponding to events occurring during the organ procurement procedure. The central server may process the received clinical timestamp data in real-time and transmit status update notifications to all designated participants. In some embodiments, this step corresponds to clinical timestamps being shared as they occur at procurement OR, wherein the central server receives timestamp signals indicating procedure milestones such as donor in OR, incision, organ visualization, and cross-clamp events, logs each timestamp in the relational database with associated organ identification information, updates the transport request status to reflect current procurement progress, and transmits real-time status update notifications to electronic devices associated with transplant coordinators, air transportation crew, and ground transportation providers to enable coordinated preparation for subsequent transportation segments.

At step 510, the method 500 may comprise calculating, by the central server, return estimated time of arrival data based on flight tracking information and ground transportation location data. The return estimated time of arrival data may comprise projected arrival times at the origin airport, projected arrival times at the recipient facility, and updated timing parameters for surgical preparation. The central server may retrieve flight tracking data from aviation data sources and aircraft transponder systems, analyze the data against stored route information, and generate refined arrival estimates for the return journey. In some embodiments, this step corresponds to ETA for the way back being shared, wherein the central server monitors return flight progress through integration with flight tracking APIs, receives real-time location data from ground transportation vehicles at the origin airport, calculates estimated arrival times at the recipient facility based on current traffic conditions and route optimization algorithms, and transmits notification signals containing the return estimated time of arrival to electronic devices associated with transplant coordinators, surgical preparation teams, and recipient facility contacts to enable coordinated preparation for organ transplantation.

In some aspects, the system may include a central server which provides the majority of processing and analysis for the organ transport logistics platform. The central server may serve as the backbone of the digital SaaS solution, coordinating the various functionalities and features that enable the automation of organ transport logistics. The central server may be hosted on AWS using a Postgres database or relational databases, thereby potentially providing scalability, reliability, and security for the processing and analysis operations.

In some cases, a frontend user interface may be retrievable by users of the system. These users may include various individuals in the organ transport chain, such as drivers, pilots, hospital staff, transplant coordinators, and charter operators. The frontend user interface may provide dedicated access to the functionalities of the system, enabling these users to communicate directly with the relevant parties, submit and review transport requests, and access historical transport data and invoicing information.

In other aspects, the central server may receive information relevant to a specific organ in transport. This information may include data such as the temperature of the organ, the time since harvest, and the estimated time to arrival at the destination. The central server may process this information in real-time, potentially enabling the system to monitor the status of the organ throughout the transportation process and ensure that the organ remains viable for transplantation.

In some instances, the central server may be able to send notifications to members of the organ transport chain. These notifications may be sent to drivers, pilots, and logistics coordinators to ensure that vehicles are available and that all parties are informed of the current status of the organ transport. The information contained in these notifications may be used to determine best routes, potentially optimizing the transportation process and reducing the time between organ retrieval and transplantation.

In some aspects, the central server may be capable of retrieving information from a node along the organ transport chain based on a request from a user of the system. This information may include real-time location data and other relevant details regarding the current position and status of the organ transport. For example, the end recipient's hospital may request real-time location information to prepare for the arrival of the organ and coordinate surgical preparations accordingly.

In some cases, charter operators or other members on the organ transport chain may edit details relevant to a given trip through the platform user interface. This functionality may allow these individuals to update information such as flight schedules, vehicle availability, estimated arrival times, and any changes to the transport plan. The ability to edit and update trip details in real-time may potentially improve the coordination and transparency of the organ transport logistics, ensuring that all parties have access to current and accurate information.

In some aspects, the system may include a scheduling module configured to manage the coordination of transportation resources for organ transport logistics. The scheduling module may consider various factors such as the availability of planes, helicopters, and ground vehicles, the urgency of the organ transport, the distance between locations, and weather conditions to generate appropriate schedules. This module may facilitate the matching of transport parameters with available resources, potentially improving the efficiency and reliability of organ transport operations.

In other aspects, the system may include a tracking module configured to provide real-time monitoring of shipments throughout the organ transportation process. The tracking module may compile shipment routes over time, building historical tracking data that transplant centers can analyze to inform future transportation preferences and decision making. This module may retrieve location information and GPS data from various nodes along the organ transport chain, enabling users to monitor the precise location and status of organs in transit.

In some instances, the system may include a chat module configured to enable secure direct messaging between all users in real-time. The chat module may facilitate communication between transplant coordinators, charter operators, drivers, pilots, and help desk staff to discuss time-sensitive organ assignments, resolve transport issues, and get assistance. This module may support the integrated chat functionality that increases transparency and coordination throughout the supply chain.

In some aspects, the system may include an insights module configured to transform historical transport data into interactive graphs and metrics for analysis and decision making. The insights module may generate visual analytics such as cost percentages, time estimates, and performance benchmarks that users can access through the dashboard. This module may process data from the relational database to provide useful insights regarding the efficiency and effectiveness of organ transport operations.

In other aspects, the system may include a proposal evaluation module configured to facilitate the review and comparison of proposals submitted by charter operators and ground transportation providers. The proposal evaluation module may present proposals to transplant centers with relevant details such as pricing, aircraft specifications, availability, and estimated travel times, enabling informed selection of preferred proposals. This module may support the workflow of receiving, reviewing, and accepting or rejecting proposals from multiple service providers.

In some instances, the system may include an invoicing module configured to streamline the billing process and aggregate financial data related to organ transport operations. The invoicing module may automate the generation and distribution of invoices to the relevant parties, such as transplant centers and charter operators, potentially reducing manual errors and administrative burden. This module may also compile and store invoicing records that users can access for financial reporting and analysis purposes.

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above described information or requesting that the user input updates or modifies existing stored information for processing. A communication subsystem may allow the methods described above to be executed or provided over a computer network. For example, the communication subsystem may be configured to enable the computing system to communicate with a plurality of personal computing devices.

The communication subsystem may include wired and/or wireless communication devices to facilitate networked communication. The described methods or processes may be executed, provided, or implemented for a user or one or more computing devices via a computer-program product such as via an application programming interface (API).

In some aspects, while the present disclosure describes the systems and methods in the context of organ transplant logistics, the underlying platform architecture, coordination mechanisms, and communication protocols may be applied to other technologies and industries requiring time-sensitive logistics coordination. The central server, matching algorithms, tracking modules, and real-time communication functionalities described herein may be adapted for use in various domains where rapid coordination of transportation resources, real-time monitoring, and multi-party communication are critical to successful outcomes.

In some aspects, the systems and methods described herein may be adapted for use in pharmaceutical logistics applications requiring time-sensitive delivery and cold chain integrity. The platform may be configured to coordinate the transportation of pharmaceuticals that require rapid delivery to medical facilities, such as antivenoms for treating patients with venomous bites or stings, where delays in administration can result in severe patient outcomes. The central server may receive transport request data from a frontend user interface, the transport request data comprising pharmaceutical type, specified temperature range parameters, storage condition specifications, origin facility location, destination hospital location, and timing parameters based on patient acuity levels. The central server may transmit the transport request data to a plurality of transportation providers based on matching criteria stored in the relational database, wherein the matching criteria comprise cold chain equipment certifications, temperature-controlled vehicle specifications, and transportation provider compliance records with pharmaceutical handling regulations. The central server may receive proposal data from the plurality of transportation providers, the proposal data comprising pricing information, vehicle specifications including refrigeration unit capabilities and temperature monitoring equipment, estimated travel times, and cold chain maintenance protocols. The scheduling module may match transport parameters with available transportation resources based on factors comprising availability of temperature-controlled vehicles, urgency of the pharmaceutical delivery based on patient condition severity, distance between the origin facility and the destination hospital, weather conditions that may impact cold chain integrity, and transportation operator certifications for pharmaceutical handling. The tracking module may be configured to retrieve temperature sensor data from monitoring devices associated with pharmaceutical shipments in real-time, wherein the temperature sensor data comprises current temperature readings, temperature history logs, and humidity measurements transmitted from electronic monitoring devices positioned within pharmaceutical transport containers. The central server may process the received temperature sensor data and generate alert notifications to members of the pharmaceutical transport chain when temperature excursions are detected that exceed predefined threshold parameters stored in the relational database, enabling rapid intervention by transportation providers to preserve product viability through activation of backup cooling systems or rerouting to alternative facilities. The chat module may enable secure direct messaging between poison control centers, hospital emergency departments, pharmacy coordinators, and transportation providers in real-time during pharmaceutical transport operations, facilitating coordination of urgent deliveries such as antivenom shipments where patient treatment timelines require precise synchronization between transportation arrival and clinical administration protocols. The insights module may transform historical pharmaceutical transport data into interactive graphs and metrics comprising temperature compliance percentages, delivery time performance benchmarks, and cold chain excursion rates for display through the frontend user interface, enabling pharmaceutical distributors and healthcare facilities to analyze transportation provider performance and inform future transportation preferences.

In other aspects, the systems and methods may be applied to pharmaceutical logistics and elements of pharmaceutical manufacturing processing. The platform may coordinate the transportation of temperature-sensitive biologics, vaccines, cell therapies, and other pharmaceutical products that require strict environmental controls during transit. The scheduling module may match transport parameters with available transportation resources based on factors comprising cold chain equipment specifications, transit time constraints, and regulatory compliance specifications. The insights module may transform historical transport data into analytics that pharmaceutical manufacturers can use to optimize their supply chain operations, identify transportation providers with superior cold chain performance, and demonstrate compliance with regulatory specifications for product integrity during distribution. The invoicing module may aggregate financial data and generate documentation specified for pharmaceutical supply chain auditing and quality assurance purposes.

In some instances, the systems and methods may be extended to time-sensitive shipment applications in industrial and aerospace contexts. The platform may coordinate the transportation of time-sensitive factory materials, industrial parts, and aerospace components that require expedited delivery to prevent manufacturing line stoppages or support aircraft maintenance operations. The central server may receive transport request data specifying part numbers, dimensional specifications, handling parameters, and delivery deadlines, and may transmit the request data to a plurality of transportation providers based on matching criteria stored in the relational database. The tracking module may retrieve real-time location data from nodes along the industrial supply chain, enabling manufacturing facilities to monitor the progress of time-sensitive shipments and adjust production schedules accordingly. The proposal evaluation module may present transportation options to procurement teams with relevant details such as pricing, transit times, and carrier reliability metrics, enabling informed selection of preferred proposals for mission-sensitive industrial logistics operations.

In some aspects, the present disclosure may relate to a computer-implemented system and method for dynamically matching, pricing, and optimizing transport resources for time-sensitive and high-acuity logistics missions. The system architecture described herein may integrate multi-source data streams to generate feasibility determinations, dynamic price estimates, ranked transport recommendations, and real-time re-optimized execution pathways. As described with reference to FIG. 1, the relational database structure comprising the trip repository 1002, participant repository 1004, organization repository 1006, user repository 1008, transportation request repository 1010, request for proposal repository 1012, and proposal repository 1014 may provide the foundational data storage infrastructure that supports the dynamic transport orchestration platform. The central server may continuously update outputs based on live data ingestion and event-driven recomputation, potentially enabling highly reliable coordination for missions where timing, compatibility, and execution certainty may be important, as illustrated in the use-case scenario 200 of FIG. 5 and the communication structure 250 of FIG. 6.

In some cases, the system may include a request intake module configured to capture mission-specific inputs through the frontend user interface. As depicted in the transportation request user interface 106 of FIG. 2, the request intake module may receive data comprising origin location, destination location, route structure, departure and arrival timing constraints, urgency classification, passenger count and escort requirements, cargo specifications including organ type and preservation method, and chain-of-custody and handling requirements. The request intake module may process these inputs and store the captured data in the transportation request repository 1010, wherein the stored data may comprise request identifiers, version numbers, construction modes, creation timestamps, equipment return indicators, air transportation preferences, expected travel parameters, organization identifiers, organ types, pickup location information, recipient facility information, recovery location information, recovery type indicators, special instructions, and transportation status indicators. The request intake module may interface with the scheduling module described herein to initiate the matching and optimization workflow upon receipt of a complete transport request.

In other aspects, the system may include a supply availability module configured to maintain a registry of available aircraft, ground vehicles, and operators within the relational database. The supply availability module may store and continuously update transportation provider profiles comprising aircraft type, tail number, category, home base and current location, crew status and duty constraints, operator preferences and minimum economics, insurance attributes, and certification records. As described with reference to the organization repository 1006, the supply availability module may maintain data points including air fleet information, insurance details, medical licensing, model specifications, power levels, special types, tail numbers, traveler capacities, vehicle identifiers, and year information. The supply availability module may interface with the telemetry ingestion module to receive real-time updates regarding aircraft positions and operational status, potentially enabling the matching algorithms to identify transportation resources that may be positioned to respond quickly to urgent transport requests. The supply availability module may maintain current data points by receiving periodic status transmissions from electronic devices associated with transportation operators, retrieving aircraft positioning data from flight tracking APIs and ADS-B receivers, and processing crew availability signals transmitted by pilots and drivers through the mobile device integration module, wherein the received data may be validated, timestamped, and stored in the relational database to ensure that the scheduling module has access to accurate and up-to-date transportation resource information when processing transport requests.

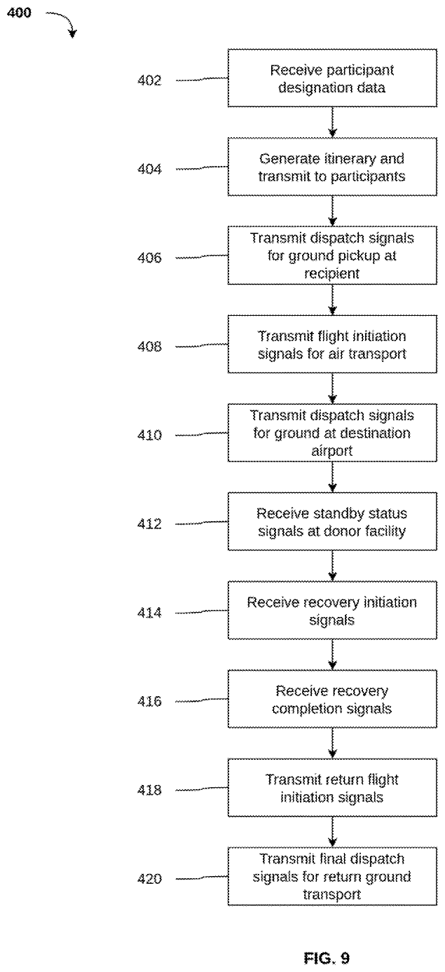

In some instances, the system may include a telemetry ingestion module configured to continuously ingest real-time operational data from multiple external sources. The telemetry ingestion module may retrieve aircraft location and movement data through integration with flight tracking APIs such as ADS-B receivers and aviation data services, as described with reference to the tracking module functionality. The telemetry ingestion module may process departure and arrival events, aircraft status indicators including in-flight, idle, and repositioning states, and estimated arrival times, storing the processed telemetry data in the relational database for access by the scheduling module and optimization engine. As described with reference to FIG. 9, the method 400 for coordinating organ transport logistics may rely upon the telemetry ingestion module to provide real-time flight tracking data during air transportation segments at steps 408 and 418, potentially enabling the central server to monitor flight progress and transmit estimated arrival notifications to ground transportation providers and facility contacts.

In some aspects, the system may include a mobile device integration module configured to ingest consent-based mobile device data from electronic devices associated with participants in the organ transport chain. As described with reference to the tracking architecture, each actor within the organ transport chain, including pilots, drivers, surgeons, perfusionists, and transplant coordinators, may be in possession of or in proximity to an electronic device with GPS capabilities that may execute a user-end version of the application or may provide location data to the central server via cellular data transmission, Wi-Fi connectivity, or satellite communication. The mobile device integration module may receive GPS location data, real-time movement and delay indicators, timestamped events including handoffs and departures, and device-derived timing validation signals. The mobile device integration module may process the received data to refine timing estimates, validate execution events, and improve chain-of-custody tracking, as illustrated in the monitoring dashboard 112 of FIG. 3 where clinical timestamps may be communicated through the messaging interface 110 during the organ procurement procedure.

In other aspects, the system may include a pricing engine configured to generate dynamic price estimates based on a hybrid pricing model that may integrate multiple computational components. The pricing engine may comprise a baseline activation model that may determine a minimum price specified to activate an aircraft based on category-based baseline parameters, operator-specific minimums stored in the organization repository 1006, and urgency-adjusted activation thresholds derived from the transport request data. The pricing engine may further comprise an incremental cost model that may apply additive cost calculations based on mission duration parameters including repositioning time, flight time, estimated duty time, and proxy variables for fuel, crew, and maintenance expenses. The pricing engine may additionally comprise a dynamic adjustment model that may apply modifiers based on urgency and time sensitivity indicators, weather conditions retrieved from external meteorological services, airport complexity factors, time-of-day effects, deadhead opportunities, and cargo handling complexity specifications. The pricing engine may output price estimates that may be stored in the proposal repository 1014 and transmitted to the frontend user interface for display through the case interface 114 depicted in FIG. 4.

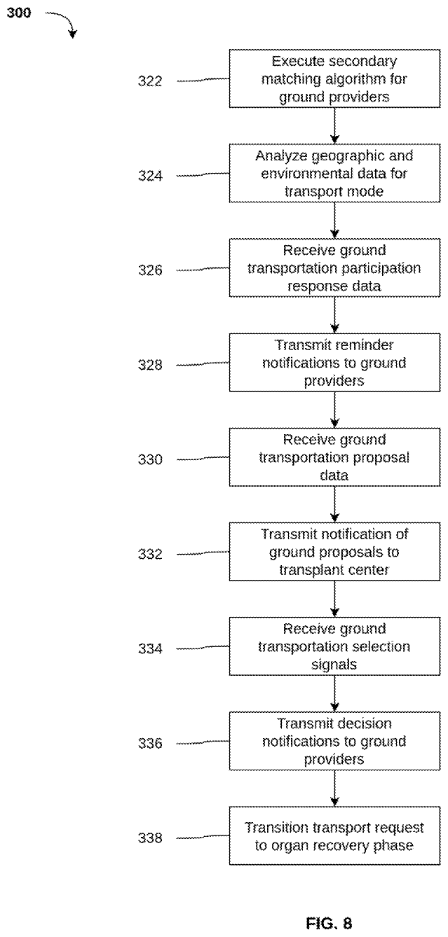

In some cases, the system may include a feasibility engine configured to evaluate whether a mission can be executed based on multiple constraint categories. The feasibility engine may perform compatibility analysis comprising range feasibility calculations, runway constraint evaluations, cargo fit assessments including volume, doorway dimensions, and payload capacity, and medical suitability determinations based on aircraft capability data stored in the organization repository 1006. The feasibility engine may further evaluate operational constraints comprising crew duty legality based on duty time parameters, aircraft positioning feasibility based on current location data received from the telemetry ingestion module, and airport access restrictions retrieved from external data sources. The feasibility engine may additionally assess clinical and service level agreement constraints comprising ability to meet specified medical timelines based on organ viability windows and operating room schedules, and probability of successful completion based on historical performance data stored in the relational database. As described with reference to step 324 of method 300 in FIG. 8, the feasibility engine may analyze geographic and environmental data to determine appropriate transportation mode configurations, including evaluating weather data, terrain data, and distance data to generate transportation mode recommendations for certain geographies with dependencies on weather conditions.

The telemetry ingestion module may interface with specific external data sources including ADS-B receivers, flight tracking APIs, and aviation data services to retrieve aircraft position data that is processed through the event stream processing layer to trigger recomputation cycles within the real-time re-optimization module, representing an implementation that meaningfully limits the scope of any underlying optimization concepts. The mobile device integration module may receive consent-based GPS location data from electronic devices associated with specific participants in the organ transport chain, processing the received data through defined computational steps to refine timing estimates and validate execution events. The pricing engine may implement a hybrid pricing model comprising baseline activation models, incremental cost models, and dynamic adjustment models that apply specific computational transformations to input parameters including repositioning time, flight time, duty time, weather conditions, and cargo handling complexity to generate price estimates stored in the proposal repository 1014. This represents a meaningful solution to the technical problem of dynamic pricing in time-sensitive logistics. Similarly, the feasibility engine may perform specific technical evaluations including range feasibility calculations, runway constraint evaluations, cargo fit assessments, and crew duty legality determinations based on data retrieved from the organization repository 1006 and the telemetry ingestion module, implementing analytical steps that maintain operation even with the volume and velocity of data inputs and the complexity of constraint interactions.

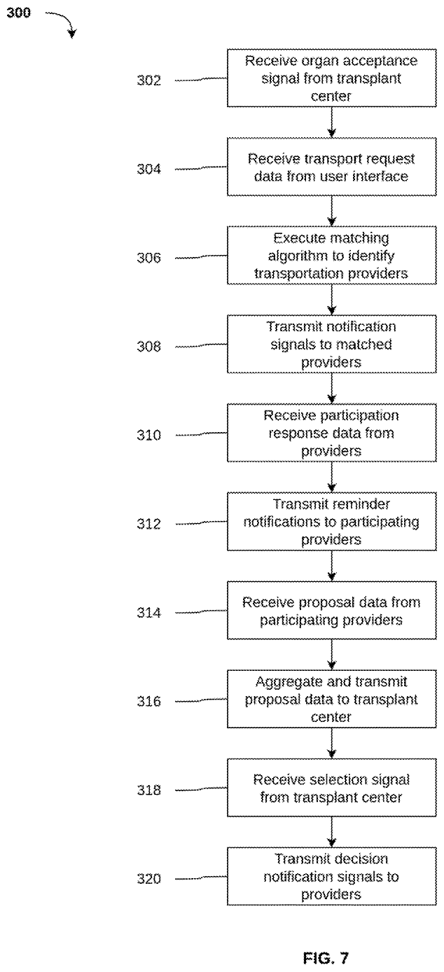

In other aspects, the system may include an optimization and ranking engine configured to balance multiple objectives and generate ranked transport recommendations. The optimization and ranking engine may implement a multi-objective optimization algorithm that may balance cost, total elapsed time, feasibility probability, and reliability score based on configurable weighting factors stored in the relational database. The optimization and ranking engine may further implement a constraint solver that may apply hard constraints comprising clinical timing parameters derived from healthcare workflow integration data, cargo compatibility specifications based on aircraft capability specifications, aircraft availability constraints based on supply availability module data, and regulatory and operational constraints based on certification and licensing records. The optimization and ranking engine may output ranked aircraft and operator options, estimated pricing, confidence levels, and alternative solutions, which may be aggregated and transmitted to the frontend user interface for display to transplant coordinators as described with reference to step 316 of method 300 in FIG. 7. The prioritization matrix implemented by the optimization and ranking engine may ensure that price does not automatically determine the selection of transportation providers, recognizing that organ viability and patient outcomes may depend substantially on reliable and safe delivery.

In some instances, the system may include a real-time re-optimization module configured to recompute recommendations dynamically when operational conditions change during mission execution. The real-time re-optimization module may monitor event streams comprising aircraft position updates from the telemetry ingestion module, operator acceptance or declination signals received through the communication subsystem, weather condition changes retrieved from external meteorological APIs, mobile device signals indicating delays or deviations from the mobile device integration module, and clinical timeline shifts communicated through the monitoring dashboard 112. Upon detection of a triggering event, the real-time re-optimization module may invoke the pricing engine, feasibility engine, and optimization and ranking engine to generate updated recommendations, which may be transmitted to the frontend user interface and to electronic devices associated with relevant participants in the organ transport chain. As described with reference to the adaptive tracking architecture, the central server may seamlessly transition between tracking schemas based on the current phase of the transport operation, automatically switching from personal electronic device tracking to flight tracking API data when aircraft take off, thereby potentially maintaining continuous and uninterrupted location monitoring that may support the real-time re-optimization functionality.

In some aspects, the system may include a learning and feedback module configured to continuously improve system performance based on historical outcome data. The learning and feedback module may compare predicted outcomes generated by the pricing engine and feasibility engine against actual outcomes recorded upon completion of transport operations, storing the comparison data in the relational database for analysis by the insights module. The learning and feedback module may update pricing parameters based on observed operator acceptance rates and actual cost outcomes, may refine feasibility scoring based on mission success and failure cases, may learn operator-specific behaviors based on historical response patterns stored in the proposal repository 1014, and may adapt optimization weights based on transplant center preferences and feedback. As described with reference to the insights module functionality, the learning and feedback module may transform historical transport data into interactive graphs and metrics comprising cost percentages, time estimates, and performance benchmarks for display through the frontend user interface, potentially enabling users to analyze transportation provider performance and inform future transportation preferences. Moreover, the learning and feedback module may compare predicted outcomes against actual outcomes and update pricing parameters, feasibility scoring, and optimization weights based on historical performance data, potentially implementing machine learning techniques in a specific technological context that may improve system performance over time.

In other aspects, the system architecture may implement an event stream processing layer configured to process real-time updates and trigger recomputation cycles within the central server. The event stream processing layer may receive aircraft telemetry updates from the telemetry ingestion module, mobile device GPS updates from the mobile device integration module, weather changes from external data integration interfaces, and mission timing updates from the healthcare workflow integration module. The event stream processing layer may evaluate each received event against configurable threshold parameters to determine whether the event warrants invocation of the real-time re-optimization module. As described with reference to the communication structure 250 of FIG. 6, the event stream processing layer may enable the central server to function as a central coordination point that may facilitate simultaneous communication with multiple transportation service providers, transmitting updated transport requests and receiving revised proposals in response to changing operational conditions. The event stream processing layer may interface with the chat module to enable secure direct messaging between transplant coordinators, charter operators, drivers, pilots, and help desk staff in real-time during organ transport operations, as depicted in the messaging interface 110 of FIG. 3.

In some aspects, the systems and methods described herein may provide improvements to the field of time-sensitive logistics coordination. As a nonlimiting example, the central server architecture, comprising the request intake module, supply availability module, telemetry ingestion module, mobile device integration module, pricing engine, feasibility engine, optimization and ranking engine, real-time re-optimization module, learning and feedback module, and/or event stream processing layer, may collectively implement a particular technological solution that improves the functioning of computer systems used to coordinate organ transport operations. In such a nonlimiting example, the system does not merely automate manual processes or apply conventional computing techniques to logistics coordination, but rather implements a processing architecture that enable real-time integration of heterogeneous data streams from aircraft telemetry systems, mobile device GPS sensors, external meteorological services, and clinical workflow systems to generate dynamically updated transport recommendations that could not be practically performed with conventional systems. The specific ordered combination of modules within the central server may transform raw telemetry data, location signals, weather information, and clinical timestamps into actionable transport coordination outputs through proprietary algorithms that analyze multiple constraint categories simultaneously, representing a concrete improvement to logistics technology. Unlike prior art systems that relied on fragmented communication channels and manual coordination processes requiring transplant coordinators to individually contact multiple charter operators and manually aggregate responses, the claimed systems and methods provide a unified technical architecture that automatically ingests real-time data from multiple heterogeneous sources, applies multi-objective optimization algorithms to generate ranked transportation recommendations, and dynamically recomputes those recommendations in response to changing operational conditions without requiring manual intervention. This technical improvement reduces latency in the coordination process from minutes or hours to seconds, preserves organ viability windows that are important to patient outcomes, and enables the processing of data volumes and computational complexity that exceed the practical capabilities of manual coordination methods or conventional logistics software.

In some aspects, the adaptive tracking architecture may seamlessly transition between tracking schemas based on the current phase of transport operations, automatically switching from personal electronic device tracking to flight tracking API data when aircraft take off, which addresses the technical problem of maintaining continuous location monitoring across heterogeneous communication environments. In some cases, the real-time re-optimization module may monitor event streams and invoke the pricing engine, feasibility engine, and optimization and ranking engine upon detection of triggering events, potentially implementing a particular feedback loop architecture that may dynamically update transport recommendations in response to changing operational conditions.

Since many modifications, variations, and changes in detail can be made to the described embodiments of the disclosure, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the disclosure should be determined by the appended claims and their legal equivalents.

In addition, the present disclosure has been described with reference to embodiments, it should be noted and understood that various modifications and variations can be crafted by those skilled in the art without departing from the scope and spirit of the disclosure. Accordingly, the foregoing disclosure should be interpreted as illustrative only and is not to be interpreted in a limiting sense. Further it is intended that any other embodiments of the present disclosure that result from any changes in application or method of use or operation, method of manufacture, shape, size, or materials which are not specified within the detailed written description or illustrations contained herein are considered within the scope of the present disclosure.

Insofar as the description above and the accompanying drawings disclose any additional subject matter that is not within the scope of the claims below, the disclosures are not dedicated to the public and the right to file one or more applications to claim such additional disclosures is reserved.

Although very narrow claims are presented herein, it should be recognized that the scope of this disclosure is much broader than presented by the claim. It is intended that broader claims will be submitted in an application that claims the benefit of priority from this application.

While this disclosure has been described with respect to at least one embodiment, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

A number of implementations have been described. Nevertheless, it will be understood that various modifications may be made without departing from the spirit and scope of the disclosure. Accordingly, other implementations are within the scope of the following claims.

1. A system for organ transport logistics, comprising:
a central server configured to coordinate organ transport operations between transplant centers and transportation providers;
a storage machine coupled to the central server and configured to store transport request data, proposal data, and historical transport data in a relational database;
a frontend user interface retrievable by users of the system, the frontend user interface configured to receive transport request inputs from transplant coordinators and display proposal information from transportation providers;
wherein the central server is configured to:
receive transport request data from the frontend user interface, the transport request data comprising organ type, recovery location, recipient facility, and timing parameters;
transmit the transport request data to a plurality of transportation providers based on matching criteria;
receive proposal data from the plurality of transportation providers, the proposal data comprising pricing information, vehicle specifications, and estimated travel times;
aggregate the proposal data and transmit the aggregated proposal data to the frontend user interface for display to the transplant coordinators;
receive a selection input indicating acceptance of a selected proposal;
retrieve real-time location data from nodes along an organ transport chain during transport operations; and
generate notifications to members of the organ transport chain based on the real-time location data.

2. The system of claim 1, wherein the central server further comprises a scheduling module configured to match transport parameters with available transportation resources based on factors comprising availability of aircraft, urgency of the organ transport, distance between the recovery location and the recipient facility, and weather conditions.

3. The system of claim 2, wherein the scheduling module is further configured to generate route recommendations based on routing optimization algorithms that analyze transplant center preferences, origin and destination locations, distances, weather data, traffic conditions, and transportation operator availability.

4. The system of claim 1, wherein the central server further comprises a tracking module configured to compile shipment routes over time and store the compiled routes as historical tracking data in the relational database for analysis by transplant centers to inform future transportation preferences.

5. The system of claim 4, wherein the tracking module is configured to retrieve real-time location data from nodes along the organ transport chain comprising ground transportation vehicles, aircraft, and donor facilities.

6. The system of claim 1, wherein the central server further comprises a chat module configured to enable secure direct messaging between transplant coordinators, charter operators, drivers, pilots, and help desk staff in real-time during organ transport operations.

7. The system of claim 1, wherein the central server further comprises an insights module configured to transform the historical transport data into interactive graphs and metrics comprising cost percentages, time estimates, and performance benchmarks for display through the frontend user interface.

8. The system of claim 1, wherein the central server further comprises an invoicing module configured to automate generation of invoices based on completed transport operations and aggregate financial data comprising billing information and payment records in the relational database.

9. A method for coordinating organ transport logistics, comprising:
receiving, by a central server, transport request data from a user interface, the transport request data comprising organ type, recovery location, recipient facility, and timing parameters;
matching, by the central server, the transport request data to a plurality of transportation providers based on matching criteria stored in a relational database;
transmitting, by the central server, the transport request data to the matched plurality of transportation providers;
receiving, by the central server, proposal data from the plurality of transportation providers, the proposal data comprising pricing information and estimated travel times;
aggregating, by the central server, the proposal data and transmitting the aggregated proposal data to the user interface;
receiving, by the central server, a selection input indicating acceptance of a selected proposal from the aggregated proposal data;
retrieving, by the central server, real-time location data from nodes along an organ transport chain during transport operations; and
generating, by the central server, notifications to members of the organ transport chain based on the retrieved real-time location data.

10. The method of claim 9, wherein matching the transport request data to the plurality of transportation providers comprises applying routing optimization algorithms that analyze transplant center preferences, origin and destination locations, distances, weather data, traffic conditions, and transportation operator availability to generate route recommendations.

11. The method of claim 10, further comprising compiling shipment routes over time and storing the compiled routes as historical tracking data in the relational database for analysis by transplant centers to inform future transportation preferences.

12. The method of claim 9, further comprising enabling secure direct messaging between transplant coordinators, charter operators, drivers, pilots, and help desk staff in real-time during the transport operations through a chat module of the central server.

13. The method of claim 9, further comprising:
transforming the historical transport data into interactive graphs and metrics comprising cost percentages, time estimates, and performance benchmarks; and
transmitting the interactive graphs and metrics to the user interface for display.

14. The method of claim 13, further comprising automating generation of invoices based on completed transport operations and aggregating financial data comprising billing information and payment records in the relational database.

15. A system for dynamically matching and optimizing transport resources for time-sensitive logistics missions, comprising:
a central server comprising:
a request intake module configured to receive mission-specific inputs through a frontend user interface, the mission-specific inputs comprising at least origin location, destination location, timing constraints, and cargo specifications;
a supply availability module configured to maintain a registry of available transportation resources and operators within a relational database, the registry comprising resource specifications, location data, and availability status;
a telemetry ingestion module configured to ingest real-time operational data from external sources, the real-time operational data comprising location data and status indicators;
a pricing engine configured to generate dynamic price estimates based on a pricing model;
a feasibility engine configured to evaluate whether a mission can be executed based on constraint categories; and
an optimization and ranking engine configured to generate ranked transport recommendations based on configurable weighting factors;
a storage machine coupled to the central server and configured to store transport data in the relational database; and
the frontend user interface retrievable by users of the system, the frontend user interface configured to receive the mission-specific inputs and display the ranked transport recommendations.

16. The system of claim 15, wherein the central server further comprises a mobile device integration module configured to ingest mobile device data from electronic devices associated with participants in a transport chain, the mobile device data comprising location data and timing information.

17. The system of claim 15, wherein the pricing engine is configured to apply a baseline activation model to determine a minimum price specified to activate a transportation resource, apply an incremental cost model based on mission duration parameters, and apply a dynamic adjustment model based on operational conditions.

18. The system of claim 15, wherein the feasibility engine is configured to perform compatibility analysis comprising capacity assessments and operational constraint evaluations based on transportation resource capability data stored in the relational database.

19. The system of claim 15, wherein the central server further comprises a real-time re-optimization module configured to monitor event streams and, upon detection of a triggering event, invoke the pricing engine, feasibility engine, and optimization and ranking engine to generate updated recommendations.

20. The system of claim 15, wherein the central server further comprises a learning and feedback module configured to compare predicted outcomes against actual outcomes recorded upon completion of transport operations, and update system parameters based on historical performance data stored in the relational database.
